SEQUENCE LISTING (96 sequences)

SEQ ID NO: 1 - protein, 5 aa, synthetic construct
  REGION 1..5: note = Synthetic: 7A3 VH CDR1 (Kabat)
GYTIH

SEQ ID NO: 2 - protein, 7 aa, synthetic construct
  REGION 1..7: note = Synthetic: 7A3 VH CDR1 (Chothia)
GYSFTGY

SEQ ID NO: 3 - protein, 17 aa, synthetic construct
  REGION 1..17: note = Synthetic: 7A3 VH CDR2 (Kabat)
LINPYTGGTDYNQKFKG

SEQ ID NO: 4 - protein, 6 aa, synthetic construct
  REGION 1..6: note = Synthetic: 7A3 VH CDR2 (Chothia)
NPYTGG

SEQ ID NO: 5 - protein, 16 aa, synthetic construct
  REGION 1..16: note = Synthetic: 7A3 VH CDR3
ERPGGSQFIYYYPMDY

SEQ ID NO: 6 - protein, 11 aa, synthetic construct
  REGION 1..11: note = Synthetic: 7A3 VL CDR1
RASANIYSYLA

SEQ ID NO: 7 - protein, 7 aa, synthetic construct
  REGION 1..7: note = Synthetic: 7A3 VL CDR2
NAITLAE

SEQ ID NO: 8 - protein, 9 aa, synthetic construct
  REGION 1..9: note = Synthetic: 7A3 VL CDR3
QHHYGTPFT

SEQ ID NO: 9 - protein, 125 aa, synthetic construct
  REGION 1..125: note = Synthetic: 7A3 VH Hu-VH2
QVQLVQSGAEVKKPGASVKVSCKASGYSFTGYTMHWVRQAPGQGLEWMGLINPYTGGTDYNQKFQGRVTMTVDKSTSTAYMELSSLRSEDTAVYYCARERPGGSQFIYYYPMDYWGQGTTVTVSS

SEQ ID NO: 10 - protein, 107 aa, synthetic construct
  REGION 1..107: note = Synthetic: 7A3 VL Hu-VL1
DIQMTQSPSSLSASVGDRVTITCRASANIYSYLAWYQQKPGKAPKFLVYNAITLAEGVPSRFSGSGSGTDFTLTISSLQPEDFATYYCQHHYGTPFTFGGGTKLEIK

SEQ ID NO: 11 - protein, 5 aa, synthetic construct
  REGION 1..5: note = Synthetic: 7B1 VH CDR1 (Kabat)
GYTMH

SEQ ID NO: 12 - protein, 7 aa, synthetic construct
  REGION 1..7: note = Synthetic: 7B1 VH CDR1 (Chothia)
GYSFTGY

SEQ ID NO: 13 - protein, 17 aa, synthetic construct
  REGION 1..17: note = Synthetic: 7B1 VH CDR2 (Kabat)
LINPYTGGTDYNQKFKG

SEQ ID NO: 14 - protein, 6 aa, synthetic construct
  REGION 1..6: note = Synthetic: 7B1 VH CDR2 (Chothia)
NPYTGG

SEQ ID NO: 15 - protein, 16 aa, synthetic construct
  REGION 1..16: note = Synthetic: 7B1 VH CDR3
ERPGGSQFIYYYALDY

SEQ ID NO: 16 - protein, 11 aa, synthetic construct
  REGION 1..11: note = Synthetic: 7B1 VL CDR1
RASENIYSYLA

SEQ ID NO: 17 - protein, 7 aa, synthetic construct
  REGION 1..7: note = Synthetic: 7B1 VL CDR2
NADTLAE

SEQ ID NO: 18 - protein, 9 aa, synthetic construct
  REGION 1..9: note = Synthetic: 7B1 VL CDR3
QHHYGTPFT

SEQ ID NO: 19 - protein, 125 aa, synthetic construct
  REGION 1..125: note = Synthetic: 7B1 VH Hu-VH2
QVQLVQSGAEVKKPGASVKVSCKASGYSFTGYTMHWVRQAPGQGLEWMGLINPYTGGTDYNQKFQGRVTMTVDRSTSTAYMELSSLRSEDTAVYYCARERPGGSQFIYYYALDYWGQGTTVTVSS

SEQ ID NO: 20 - protein, 107 aa, synthetic construct
  REGION 1..107: note = Synthetic: 7B1 VL Hu-VL1
DIQMTQSPSSLSASVGDRVTITCRASENIYSYLAWYQQKPGKAPKFLVYNADTLAEGVPSRFSGSGSGTDFTLTISSLQPEDFATYYCQHHYGTPFTFGGGTKLEIK

SEQ ID NO: 21 - protein, 5 aa, synthetic construct
  REGION 1..5: note = Synthetic: VH CDR1 Consensus
  VARIANT 4: note = X is I or M
GYTXH

SEQ ID NO: 22 - protein, 16 aa, synthetic construct
  REGION 1..16: note = Synthetic: VH CDR3 Consensus
  VARIANT 13: note = X is P or A
  VARIANT 14: note = X is M or L
ERPGGSQFIYYYXXDY

SEQ ID NO: 23 - protein, 11 aa, synthetic construct
  REGION 1..11: note = Synthetic: VL CDR1 Consensus
  VARIANT 4: note = X is A or E
RASXNIYSYLA

SEQ ID NO: 24 - protein, 7 aa, synthetic construct
  REGION 1..7: note = Synthetic: VL CDR2 Consensus
  VARIANT 3: note = X is I or D
NAXTLAE

SEQ ID NO: 25 - protein, 454 aa, synthetic construct
  REGION 1..454: note = Synthetic: 7A3 AQQ HC amino acids
QVQLVQSGAEVKKPGASVKVSCKASGYSFTGYTMHWVRQAPGQGLEWMGLINPYTGGTDYNQKFQGRVTMTVDKSTSTAYMELSSLRSEDTAVYYCARERPGGSQFIYYYPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 214 aa, synthetic construct
  REGION 1..214: note = Synthetic: 7A3 LC amino acids
DIQMTQSPSSLSASVGDRVTITCRASANIYSYLAWYQQKPGKAPKFLVYNAITLAEGVPSRFSGSGSGTDFTLTISSLQPEDFATYYCQHHYGTPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 454 aa, synthetic construct
  REGION 1..454: note = Synthetic: 7B1 AQQ HC amino acids
QVQLVQSGAEVKKPGASVKVSCKASGYSFTGYTMHWVRQAPGQGLEWMGLINPYTGGTDYNQKFQGRVTMTVDRSTSTAYMELSSLRSEDTAVYYCARERPGGSQFIYYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 214 aa, synthetic construct
  REGION 1..214: note = Synthetic: 7B1 LC amino acids
DIQMTQSPSSLSASVGDRVTITCRASENIYSYLAWYQQKPGKAPKFLVYNADTLAEGVPSRFSGSGSGTDFTLTISSLQPEDFATYYCQHHYGTPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 7 aa, synthetic construct
  REGION 1..7: note = Synthetic: VH CDR - PD1-HuAb-E1A9C8A7-V8-3
GYTFTDY

SEQ ID NO: 30 - protein, 10 aa, synthetic construct
  REGION 1..10: note = Synthetic: VH CDR - PD1-HuAb-E1A9C8A7-V8-3
GYTFTDYVMH

SEQ ID NO: 31 - protein, 5 aa, synthetic construct
  REGION 1..5: note = Synthetic: VH CDR - PD1-HuAb-E1A9C8A7-V8-3
DYVMH

SEQ ID NO: 32 - protein, 6 aa, synthetic construct
  REGION 1..6: note = Synthetic: VH CDR - PD1-HuAb-E1A9C8A7-V8-3
TDYVMH

SEQ ID NO: 33 - protein, 8 aa, synthetic construct
  REGION 1..8: note = Synthetic: VH CDR - PD1-HuAb-E1A9C8A7-V8-3
GYTFTDYV

SEQ ID NO: 34 - protein, 6 aa, synthetic construct
  REGION 1..6: note = Synthetic: VH CDR - PD1-HuAb-E1A9C8A7-V8-3
STYHIP

SEQ ID NO: 35 - protein, 10 aa, synthetic construct
  REGION 1..10: note = Synthetic: VH CDR - PD1-HuAb-E1A9C8A7-V8-3
VISTYHIPAV

SEQ ID NO: 36 - protein, 17 aa, synthetic construct
  REGION 1..17: note = Synthetic: VH CDR - PD1-HuAb-E1A9C8A7-V8-3
VISTYHIPAVYNQKFKG

SEQ ID NO: 37 - protein, 13 aa, synthetic construct
  REGION 1..13: note = Synthetic: VH CDR - PD1-HuAb-E1A9C8A7-V8-3
WMGVISTYHIPAV

SEQ ID NO: 38 - protein, 8 aa, synthetic construct
  REGION 1..8: note = Synthetic: VH CDR - PD1-HuAb-E1A9C8A7-V8-3
ISTYHIPA

SEQ ID NO: 39 - protein, 11 aa, synthetic construct
  REGION 1..11: note = Synthetic: VH CDR - PD1-HuAb-E1A9C8A7-V8-3
EVYGESYYFDV

SEQ ID NO: 40 - protein, 11 aa, synthetic construct
  REGION 1..11: note = Synthetic: VH CDR - PD1-HuAb-E1A9C8A7-V8-3
EVYGESYYFDV

SEQ ID NO: 41 - protein, 11 aa, synthetic construct
  REGION 1..11: note = Synthetic: VH CDR - PD1-HuAb-E1A9C8A7-V8-3
EVYGESYYFDV

SEQ ID NO: 42 - protein, 12 aa, synthetic construct
  REGION 1..12: note = Synthetic: VH CDR - PD1-HuAb-E1A9C8A7-V8-3
AREVYGESYYFD

SEQ ID NO: 43 - protein, 13 aa, synthetic construct
  REGION 1..13: note = Synthetic: VH CDR - PD1-HuAb-E1A9C8A7-V8-3
AREVYGESYYFDV

SEQ ID NO: 44 - protein, 16 aa, synthetic construct
  REGION 1..16: note = Synthetic: VL CDR - PD1-HuAb-E1A9C8A7-V8-3
RSSQSIVHSAGNTYLE

SEQ ID NO: 45 - protein, 16 aa, synthetic construct
  REGION 1..16: note = Synthetic: VL CDR - PD1-HuAb-E1A9C8A7-V8-3
RSSQSIVHSAGNTYLE

SEQ ID NO: 46 - protein, 16 aa, synthetic construct
  REGION 1..16: note = Synthetic: VL CDR - PD1-HuAb-E1A9C8A7-V8-3
RSSQSIVHSAGNTYLE

SEQ ID NO: 47 - protein, 12 aa, synthetic construct
  REGION 1..12: note = Synthetic: VL CDR - PD1-HuAb-E1A9C8A7-V8-3
VHSAGNTYLEWY

SEQ ID NO: 48 - protein, 11 aa, synthetic construct
  REGION 1..11: note = Synthetic: VL CDR - PD1-HuAb-E1A9C8A7-V8-3
QSIVHSAGNTY

SEQ ID NO: 49 - protein, 7 aa, synthetic construct
  REGION 1..7: note = Synthetic: VL CDR - PD1-HuAb-E1A9C8A7-V8-3
KVSNRFS

SEQ ID NO: 50 - protein, 7 aa, synthetic construct
  REGION 1..7: note = Synthetic: VL CDR - PD1-HuAb-E1A9C8A7-V8-3
KVSNRFS

SEQ ID NO: 51 - protein, 7 aa, synthetic construct
  REGION 1..7: note = Synthetic: VL CDR - PD1-HuAb-E1A9C8A7-V8-3
KVSNRFS

SEQ ID NO: 52 - protein, 10 aa, synthetic construct
  REGION 1..10: note = Synthetic: VL CDR - PD1-HuAb-E1A9C8A7-V8-3
LLIYKVSNRF

SEQ ID NO: 53 - skipped

SEQ ID NO: 54 - protein, 9 aa, synthetic construct
  REGION 1..9: note = Synthetic: VL CDR - PD1-HuAb-E1A9C8A7-V8-3
FQGSHVPYT

SEQ ID NO: 55 - protein, 9 aa, synthetic construct
  REGION 1..9: note = Synthetic: VL CDR - PD1-HuAb-E1A9C8A7-V8-3
FQGSHVPYT

SEQ ID NO: 56 - protein, 9 aa, synthetic construct
  REGION 1..9: note = Synthetic: VL CDR - PD1-HuAb-E1A9C8A7-V8-3
FQGSHVPYT

SEQ ID NO: 57 - protein, 8 aa, synthetic construct
  REGION 1..8: note = Synthetic: VL CDR - PD1-HuAb-E1A9C8A7-V8-3
FQGSHVPY

SEQ ID NO: 58 - protein, 9 aa, synthetic construct
  REGION 1..9: note = Synthetic: VL CDR - PD1-HuAb-E1A9C8A7-V8-3
FQGSHVPYT

SEQ ID NO: 59 - protein, 120 aa, synthetic construct
  REGION 1..120: note = Synthetic: Amino Acid Sequence (PD1-HuAb-E1A9C8A7-V8-3)
QVQLVQSGAEVVKPGASVKISCKASGYTFTDYVMHWVRQAPGQSLEWMGVISTYHIPAVYNQKFKGKATMTVDTSTSTVYLELSSLRSEDTAVYYCAREVYGESYYFDVWGQGTTVTVSS

SEQ ID NO: 60 - protein, 112 aa, synthetic construct
  REGION 1..112: note = Synthetic: Amino Acid Sequence (PD1-HuAb-E1A9C8A7-V8-3)
DIVMTQTPLSLPVTPGEPASISCRSSQSIVHSAGNTYLEWYLQKPGQSPQLLIYKVSNRFSGVPDRFSGSGSGTDFTLKISRVEAEDVGVYYCFQGSHVPYTFGGGTKVEIK

SEQ ID NO: 61 - protein, 120 aa, synthetic construct
  REGION 1..120: note = Synthetic: Amino Acid Sequence (PD1-HuAb-E1A9C8A7-V8)
QVQLVQSGAEVVKPGASVKISCKASGYTFTDYVMHWVRQAPGQSLEWMGVISTYHIPAVYNQKFKGKATMTVDTSTSTVYLELSSLRSEDTAVYYCAREVYGDSYYFDVWGQGTTVTVSS

SEQ ID NO: 62 - protein, 112 aa, synthetic construct
  REGION 1..112: note = Synthetic: Amino Acid Sequence (PD1-HuAb-E1A9C8A7-V8)
DIVMTQTPLSLPVTPGEPASISCRSSQSIVHSNGNTYLEWYLQKPGQSPQLLIYKVSNRFSGVPDRFSGSGSGTDFTLKISRVEAEDVGVYYCFQGSHVPYTFGGGTKVEIK

SEQ ID NO: 63 - protein, 219 aa, synthetic construct
  REGION 1..219: note = Synthetic: CDX-585 LC amino acids
DIVMTQTPLSLPVTPGEPASISCRSSQSIVHSAGNTYLEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 710 aa, synthetic construct
  REGION 1..710: note = Synthetic: CDX-585 HC amino acids
QVQLVQSGAEVVKPGASVKISCKASGYTFTDYVMHWVRQAPGQSLEWMGVISTYHIPAVYNQKFKGKATMTVDTSTSTVYLELSSLRSEDTAVYYCAREVYGESYYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGGGSDIQMTQSPSSLSASVGDRVTITCRASENIYSYLAWYQQKPGKAPKFLVYNADTLAEGVPSRFSGSGSGTDFTLTISSLQPEDFATYYCQHHYGTPFTFGCGTKLEIKGGGGSGGGGSGGGGSGGGGSQVQLVQSGAEVKKPGASVKVSCKASGYSFTGYTMHWVRQAPGQCLEWMGLINPYTGGTDYNQKFQGRVTMTVDRSTSTAYMELSSLRSEDTAVYYCARERPGGSQFIYYYALDYWGQGTTVTVSS

SEQ ID NO: 65 - protein, 660 aa, synthetic construct
  REGION 1..660: note = Synthetic: E1A9-7B1 (CDX-585) LC nucleic acids
GACATCGTGATGACCCAGACACCATTGTCGTTGCCTGTTACTCCAGGAGAACCCGCATCAATTAGCTGCAGAAGCAGCCAGTCCATTGTGCATAGTGCCGGTAACACCTATCTGGAGTGGTACCTACAGAAGCCCGGCCAGTCTCCTCAGCTTCTTATCTACAAAGTGAGCAATAGGTTTTCTGGGGTGCCTGATCGGTTTAGCGGTAGCGGCTCCGGCACCGACTTTACACTGAAGATCAGCAGGGTGGAGGCTGAGGACGTTGGAGTGTACTACTGTTTTCAGGGCAGCCACGTGCCTTATACATTTGGTGGCGGCACAAAGGTCGAGATCAAACGTACGGTGGCCGCTCCAAGCGTGTTCATCTTTCCCCCTTCTGACGAGCAGCTGAAGTCTGGCACAGCCTCCGTGGTGTGCCTGCTGAACAACTTCTACCCCAGAGAGGCCAAGGTGCAGTGGAAGGTGGATAACGCTCTGCAGTCTGGCAATTCCCAGGAGAGCGTGACCGAGCAGGACTCTAAGGATTCCACATATAGCCTGAGCTCTACCCTGACACTGTCTAAGGCCGATTACGAGAAGCACAAGGTGTATGCTTGCGAGGTGACCCATCAGGGCCTGTCCAGCCCAGTGACAAAGTCCTTCAATCGCGGCGAGTGTTGA

SEQ ID NO: 66 - protein, 2133 aa, synthetic construct
  REGION 1..2133: note = Synthetic: E1A9-7B1 (CDX-585) HC nucleic acids
CAAGTACAGCTCGTGCAATCTGGCGCCGAGGTAGTTAAACCTGGAGCATCCGTCAAAATTAGCTGTAAGGCAAGTGGCTACACTTTCACCGATTACGTCATGCACTGGGTGCGGCAGGCCCCCGGACAGAGTCTGGAGTGGATGGGCGTAATATCAACCTACCATATACCCGCCGTGTACAATCAGAAATTTAAGGGTAAGGCAACCATGACCGTTGACACCTCCACCTCTACAGTCTATCTGGAATTGTCTTCCCTAAGATCAGAGGACACTGCCGTCTACTACTGTGCCCGCGAAGTTTATGGAGAGTCATATTACTTCGATGTTTGGGGTCAGGGAACTACAGTAACTGTGAGCAGCGCTAGCACAAAGGGCCCTTCCGTGTTTCCACTGGCTCCCAGCTCTAAGTCCACCAGCGGAGGAACAGCCGCTCTGGGCTGTCTGGTGAAGGACTATTTCCCAGAGCCCGTGACCGTGAGCTGGAACTCTGGCGCCCTGACCAGCGGAGTGCATACATTTCCTGCTGTGCTGCAGTCCAGCGGCCTGTACTCTCTGTCTTCCGTGGTGACCGTGCCAAGCTCTTCCCTGGGCACCCAGACATATATCTGCAACGTGAATCACAAGCCATCCAATACAAAGGTGGACAAGAAGGTGGAGCCCAAGAGCTGTGATAAGACCCATACATGCCCCCCTTGTCCTGCTCCAGAGGCTCAGGGAGGACCATCCGTGTTCCTGTTTCCACCCAAGCCTAAGGACACCCTGTACATCACAAGGGAGCCAGAGGTGACCTGCGTGGTGGTGGACGTGAGCCACGAGGATCCCGAGGTGAAGTTCAACTGGTACGTGGATGGCGTGGAGGTGCATAATGCCAAGACAAAGCCAAGGGAGGAGCAGTACAATAGCACCTATCGGGTGGTGTCTGTGCTGACAGTGCTGCACCAGGACTGGCTGAACGGCAAGGAGTACAAGTGCCAGGTGTCTAATAAGGCCCTGCCCGCTCCTATCGAGAAGACCATCTCCAAGGCCAAGGGCCAGCCTAGGGAGCCACAGGTGTACACACTGCCTCCAAGCCGGGACGAGCTGACCAAGAACCAGGTGTCTCTGACATGTCTGGTGAAGGGCTTCTATCCCTCTGATATCGCTGTGGAGTGGGAGTCCAATGGCCAGCCTGAGAACAATTACAAGACCACACCCCCTGTGCTGGACTCCGATGGCAGCTTCTTTCTGTATTCCAAGCTGACCGTGGATAAGAGCAGGTGGCAGCAGGGCAACGTGTTTTCTTGTTCCGTGATGCATGAGGCTCTGCACAATCATTACACACAGAAGAGCCTGTCTCTGTCCCCTGGCAAAGGCTCGAGCGGGGGAGGAGGTAGCGACATCCAGATGACACAGAGCCCAAGCTCTCTGAGCGCCAGCGTGGGAGATAGGGTGACAATCACTTGTAGGGCCAGCGAGAACATCTACAGCTATCTGGCTTGGTACCAGCAGAAGCCCGGCAAGGCCCCAAAGTTTCTGGTGTACAACGCCGACACTCTCGCTGAGGGCGTCCCTTCTAGGTTTTCCGGCAGCGGCTCCGGCACTGACTTCACACTGACTATCAGCTCTCTGCAGCCAGAGGATTTCGCCACATACTACTGCCAGCACCACTACGGCACTCCTTTCACATTCGGCTGCGGCACTAAGCTGGAGATCAAGGGAGGGGGCGGTTCCGGAGGAGGCGGCAGCGGGGGAGGAGGTAGCGGCGGAGGTGGGTCTCAAGTGCAGCTGGTGCAGAGCGGCGCTGAGGTGAAGAAGCCCGGCGCCAGCGTGAAAGTGAGCTGTAAGGCCTCCGGCTACAGCTTCACTGGCTACACTATGCACTGGGTCAGACAAGCCCCCGGCCAATGCCTGGAGTGGATGGGACTGATCAACCCTTACACTGGCGGCACTGACTACAACCAGAAGTTCCAAGGAAGGGTGACTATGACTGTGGATAGGTCCACAAGCACAGCCTACATGGAGCTGTCCTCTCTGAGATCCGAGGACACTGCCGTGTACTACTGTGCTAGGGAGAGACCCGGCGGCAGCCAGTTCATCTACTACTACGCTCTGGACTACTGGGGCCAAGGCACAACAGTCACAGTGAGCAGCTGA

SEQ ID NO: 67 - protein, 7 aa, synthetic construct
  REGION 1..7: note = Synthetic: VH CDR - PD1-HuAb-E1A9C8A7-V8
GYTFTDY

SEQ ID NO: 68 - protein, 10 aa, synthetic construct
  REGION 1..10: note = Synthetic: VH CDR - PD1-HuAb-E1A9C8A7-V8
GYTFTDYVMH

SEQ ID NO: 69 - protein, 5 aa, synthetic construct
  REGION 1..5: note = Synthetic: VH CDR - PD1-HuAb-E1A9C8A7-V8
DYVMH

SEQ ID NO: 70 - protein, 6 aa, synthetic construct
  REGION 1..6: note = Synthetic: VH CDR - PD1-HuAb-E1A9C8A7-V8
TDYVMH

SEQ ID NO: 71 - protein, 8 aa, synthetic construct
  REGION 1..8: note = Synthetic: VH CDR - PD1-HuAb-E1A9C8A7-V8
GYTFTDYV

SEQ ID NO: 72 - protein, 6 aa, synthetic construct
  REGION 1..6: note = Synthetic: VH CDR - PD1-HuAb-E1A9C8A7-V8
STYHIP

SEQ ID NO: 73 - protein, 10 aa, synthetic construct
  REGION 1..10: note = Synthetic: VH CDR - PD1-HuAb-E1A9C8A7-V8
VISTYHIPAV

SEQ ID NO: 74 - protein, 17 aa, synthetic construct
  REGION 1..17: note = Synthetic: VH CDR - PD1-HuAb-E1A9C8A7-V8
VISTYHIPAVYNQKFKG

SEQ ID NO: 75 - protein, 13 aa, synthetic construct
  REGION 1..13: note = Synthetic: VH CDR - PD1-HuAb-E1A9C8A7-V8
WMGVISTYHIPAV

SEQ ID NO: 76 - protein, 8 aa, synthetic construct
  REGION 1..8: note = Synthetic: VH CDR - PD1-HuAb-E1A9C8A7-V8
ISTYHIPA

SEQ ID NO: 77 - protein, 11 aa, synthetic construct
  REGION 1..11: note = Synthetic: VH CDR - PD1-HuAb-E1A9C8A7-V8
EVYGDSYYFDV

SEQ ID NO: 78 - protein, 11 aa, synthetic construct
  REGION 1..11: note = Synthetic: VH CDR - PD1-HuAb-E1A9C8A7-V8
EVYGDSYYFDV

SEQ ID NO: 79 - protein, 11 aa, synthetic construct
  REGION 1..11: note = Synthetic: VH CDR - PD1-HuAb-E1A9C8A7-V8
EVYGDSYYFDV

SEQ ID NO: 80 - protein, 12 aa, synthetic construct
  REGION 1..12: note = Synthetic: VH CDR - PD1-HuAb-E1A9C8A7-V8
AREVYGDSYYFD

SEQ ID NO: 81 - protein, 13 aa, synthetic construct
  REGION 1..13: note = Synthetic: VH CDR - PD1-HuAb-E1A9C8A7-V8
AREVYGDSYYFDV

SEQ ID NO: 82 - protein, 16 aa, synthetic construct
  REGION 1..16: note = Synthetic: VL CDR - PD1-HuAb-E1A9C8A7-V8
RSSQSIVHSNGNTYLE

SEQ ID NO: 83 - protein, 16 aa, synthetic construct
  REGION 1..16: note = Synthetic: VL CDR - PD1-HuAb-E1A9C8A7-V8
RSSQSIVHSNGNTYLE

SEQ ID NO: 84 - protein, 16 aa, synthetic construct
  REGION 1..16: note = Synthetic: VL CDR - PD1-HuAb-E1A9C8A7-V8
RSSQSIVHSNGNTYLE

SEQ ID NO: 85 - protein, 12 aa, synthetic construct
  REGION 1..12: note = Synthetic: VL CDR - PD1-HuAb-E1A9C8A7-V8
VHSNGNTYLEWY

SEQ ID NO: 86 - protein, 11 aa, synthetic construct
  REGION 1..11: note = Synthetic: VL CDR - PD1-HuAb-E1A9C8A7-V8
QSIVHSNGNTY

SEQ ID NO: 87 - protein, 7 aa, synthetic construct
  REGION 1..7: note = Synthetic: VL CDR - PD1-HuAb-E1A9C8A7-V8
KVSNRFS

SEQ ID NO: 88 - protein, 7 aa, synthetic construct
  REGION 1..7: note = Synthetic: VL CDR - PD1-HuAb-E1A9C8A7-V8
KVSNRFS

SEQ ID NO: 89 - protein, 7 aa, synthetic construct
  REGION 1..7: note = Synthetic: VL CDR - PD1-HuAb-E1A9C8A7-V8
KVSNRFS

SEQ ID NO: 90 - protein, 10 aa, synthetic construct
  REGION 1..10: note = Synthetic: VL CDR - PD1-HuAb-E1A9C8A7-V8
LLIYKVSNRF

SEQ ID NO: 91 - skipped

SEQ ID NO: 92 - protein, 9 aa, synthetic construct
  REGION 1..9: note = Synthetic: VL CDR - PD1-HuAb-E1A9C8A7-V8
FQGSHVPYT

SEQ ID NO: 93 - protein, 9 aa, synthetic construct
  REGION 1..9: note = Synthetic: VL CDR - PD1-HuAb-E1A9C8A7-V8
FQGSHVPYT

SEQ ID NO: 94 - protein, 9 aa, synthetic construct
  REGION 1..9: note = Synthetic: VL CDR - PD1-HuAb-E1A9C8A7-V8
FQGSHVPYT

SEQ ID NO: 95 - protein, 8 aa, synthetic construct
  REGION 1..8: note = Synthetic: VL CDR - PD1-HuAb-E1A9C8A7-V8
FQGSHVPY

SEQ ID NO: 96 - protein, 9 aa, synthetic construct
  REGION 1..9: note = Synthetic: VL CDR - PD1-HuAb-E1A9C8A7-V8
FQGSHVPYT

(19) United States
(12) Patent Application Publication
(10) Pub. No.: US 2024/0409639 A1
(43) Pub. Date: Dec. 12, 2024

(54) ANTI-ILT4 AND ANTI-PD-1 BISPECIFIC CONSTRUCTS

(71) Applicants: Celldex Therapeutics, Inc., Hampton, NJ (US); Biosion, Inc., Jiangsu (CN)

(72) Inventors: Tibor KELER, Pipersville, PA (US); Joel GOLDSTEIN, Hopewell, NJ (US); Michael MURPHY, Wallingford, CT (US); Thomas O'NEILL, Washington, NJ (US); Laura A. VITALE, Doylestown, PA (US); Mingjiu CHEN, Jiangsu (CN)

(21) Appl. No.: 18/698,680

(22) PCT Filed: Nov. 8, 2022
(86) PCT No.: PCT/US2022/079442

§ 371 Date: Apr. 4, 2024

(30) Foreign Application Priority Data
Nov. 8, 2021 (WO) PCT/CN2021/129380

(51) Int. Cl. C07K 16/28; A61K 39/00; A61P 35/00

(52) U.S. Cl. CPC C07K 16/2818; A61P 35/00; A61K 2039/507; C07K 2317/31; C07K 2317/565; C07K 2317/622; C07K 2317/732; C07K 2317/76; C07K 2317/92

(57) ABSTRACT

Provided herein are novel anti-ILT4 and anti-PD-1 bispecific constructs, as well as corresponding compositions. Meth-ods of inducing or enhancing an immune response, and methods of treating cancer, by administering the constructs or compositions also are described.

### CROSS-REFERENCE TO RELATED APPLICATIONS

This application claims priority under 35 U.S.C. § 119 to International Application No. PCT/CN2021/129380, filed Nov. 8, 2021. The contents of the aforementioned application are hereby incorporated by reference.

### I. BACKGROUND OF THE INVENTION

The inhibitory immune checkpoint receptor “immunoglobulin-like transcript 4” (ILT4) is a member of the non-catalytic tyrosine-phosphorylated receptor family which is expressed on immune cells (such as T cells, B cells, NK cells, dendritic cells, macrophages and mast cells). Like other receptors in this family, ILT4 contains a conserved sequence of amino acids in the cytoplasmic domain referred to as an immunoreceptor tyrosine-based inhibitory motif (ITIM). (Veillette et al. (2002) Annual Review of Immunology 20 (1): 669-707). Binding and activation of ILT4 by its cognate ligands (HLA-G and HLA Class I in myeloid cells) has immunosuppressive effects through multiple mechanisms. ILT4 is also found in tumor cells and stroma cells in the tumor microenvironment of various cancers and has been shown to modulate the biological behaviors of tumor cells, thus promoting their immune escape. (Gao et al. (2018) Biochimica et Biophysica Acta (BBA)—Reviews on Cancer 1869 (2): 278-285). Accordingly, the expression of ILT4 in several tumor types is associated with poor outcome.

Programmed cell death protein 1 (PD-1) is a cell surface receptor that belongs to the immunoglobulin superfamily and is expressed on T cells and pro-B cells. PD-1 binds two ligands, PD-L1 and PD-L2. PD-1 regulates the immune system's response to the cells of the human body by down-regulating the immune system and promoting self-tolerance by suppressing T cell inflammatory activity. This prevents autoimmune diseases, but it can also prevent the immune system from killing cancer cells. PD-1 is an immune checkpoint and guards against autoimmunity through two mechanisms; (a) it promotes apoptosis (programmed cell death) of antigen-specific T-cells in lymph nodes and (b) it reduces apoptosis in regulatory T cells (anti-inflammatory, suppressive T cells). Immune suppression can be reversed by inhibiting PD-1.

Despite advances associated with antibody therapy, there is a need in the art for new and improved therapeutic agents to treat conditions or diseases, e.g., in which stimulation of an immune response is desired. Accordingly, it is an object of the present invention to provide improved methods for treating subjects with such conditions or diseases, such as cancer.

### II. SUMMARY OF THE INVENTION

Provided herein are novel bispecific constructs comprising an anti-ILT4 binding domain linked to an anti-PD-1 binding domain. As further described herein, the bispecific constructs of the present invention can be used in methods of inducing or enhancing an immune response and methods of treating a disease or condition (e.g., cancer).

In one embodiment, the bispecific construct comprises an anti-ILT4 binding domain linked to an anti-PD-1 binding domain, wherein
(a) the ILT4 binding domain comprises heavy and light chain CDR1, CDR2 and CDR3 domains having the following sequences:
   (i) a heavy chain variable region CDR1 amino acid sequence selected from the consensus sequence: G Y T (I,M) H (SEQ ID NO: 21), or conservative sequence modifications thereof;
   (ii) a heavy chain variable region CDR2 amino acid sequence as set forth in SEQ ID NO:3, or conservative sequence modifications thereof;
   (iii) a heavy chain variable region CDR3 amino acid sequence selected from the consensus sequence: E R P G G S Q F I Y Y Y (P,A) (M,L) D Y (SEQ ID NO:22), or conservative sequence modifications thereof;
   (iv) a light chain variable region CDR1 amino acid sequence selected from the consensus sequence: R A S (A,E) N I Y S Y L A (SEQ ID NO: 23), or conservative sequence modifications thereof;
   (v) a light chain variable region CDR2 amino acid sequence selected from the consensus sequence: N A (I,D) T L A E (SEQ ID NO: 24), or conservative sequence modifications thereof;
   (vi) a light chain variable region CDR3 amino acid sequence as set forth in SEQ ID NO:8, or conservative sequence modifications thereof; and
(b) the anti-PD-1 binding domain comprises:
   (i) heavy chain variable region CDR1, CDR2 and CDR3 amino acid sequences as set forth in SEQ ID NOs: 31, 36, and 41, respectively, or conservative sequence modifications thereof, and light chain variable region CDR1, CDR2 and CDR3 amino acid sequences as set forth in SEQ ID NOs: 46, 51, and 56, respectively, or conservative sequence modifications thereof; or
   (ii) the anti-PD-1 binding domain comprises heavy chain variable region CDR1, CDR2 and CDR3 as set forth in SEQ ID NOs: 69, 74, and 79, respectively, or conservative sequence modifications thereof, and light chain variable region CDR1, CDR2 and CDR3 as set forth in SEQ ID NOs: 84, 89, and 94, respectively, or conservative sequence modifications thereof, and a human IgG1 constant domain.

In another embodiment, the anti-ILT4 binding domain comprises:
(a) heavy chain variable region CDR1, CDR2 and CDR3 amino acid sequences as set forth in SEQ ID NOs: 11, 13, and 15, respectively, or conservative sequence modifications thereof, and light chain variable region CDR1, CDR2 and CDR3 amino acid sequences as set forth in SEQ ID NOs: 16, 17, and 18, respectively, or conservative sequence modifications thereof, or
(b) heavy chain variable region CDR1, CDR2 and CDR3 amino acid sequences as set forth in SEQ ID NOs: 1, 3, and 5, respectively, or conservative sequence modifications thereof, and light chain variable region CDR1, CDR2 and CDR3 amino acid sequences as set forth in SEQ ID NOs: 10, 11, and 12, respectively, or conservative sequence modifications thereof; and light chain variable region CDR1, CDR2 and CDR3 amino acid sequences as set forth in SEQ ID NOs: 6, 7, and 8, respectively, or conservative sequence modifications thereof.

In another embodiment, the bispecific construct comprises an anti-ILT4 binding domain linked to an anti-PD-1 binding domain, wherein:
(a) the anti-ILT4 binding domain comprises heavy chain variable region CDR1, CDR2 and CDR3 amino acid sequences as set forth in SEQ ID NOs: 11, 13, and 15, respectively, and light chain variable region CDR1, CDR2 and CDR3 amino acid sequences as set forth in SEQ ID NOs: 16, 17, and 18, respectively; and
(b) the anti-PD-1 binding domain comprises heavy chain variable region CDR1, CDR2 and CDR3 amino acid sequences as set forth in SEQ ID NOs: 31, 36, and 41, respectively, and light chain variable region CDR1, CDR2 and CDR3 amino acid sequences as set forth in SEQ ID NOs: 46, 51, and 56, respectively.

In yet another embodiment, the anti-ILT4 binding domain of the bispecific construct comprises:
(a) a heavy chain variable region amino acid sequence as set forth in SEQ ID NO:19, or a sequence at least 95% identical thereto, and a light chain variable region amino acid sequence as set forth in SEQ ID NO:20, or a sequence at least 95% identical thereto; or
(b) a heavy chain variable region amino acid sequence as set forth in SEQ ID NO:9, or a sequence at least 95% identical thereto, and a light chain variable region amino acid sequence as set forth in SEQ ID NO: 10, or a sequence at least 95% identical thereto.

In yet another embodiment, the anti-PD-1 binding domain of the bispecific construct comprises:
(a) a heavy chain variable region amino acid sequence as set forth in SEQ ID NO:59, or a sequence at least 95% identical thereto, and a light chain variable region amino acid sequence as set forth in SEQ ID NO:60, or a sequence at least 95% identical thereto; or.
(b) a heavy chain variable region amino acid sequence as set forth in SEQ ID NO:61, or a sequence at least 95% identical thereto, and a light chain variable region amino acid sequence as set forth in SEQ ID NO:62, or a sequence at least 95% identical thereto.

In another embodiment, the anti-ILT4 binding domain of the bispecific construct comprises a heavy chain variable region amino acid sequence as set forth in SEQ ID NO:19 and a light chain variable region amino acid sequence as set forth in SEQ ID NO:20. Alternatively, the anti-ILT4 binding domain of the bispecific construct comprises a heavy chain variable region amino acid sequence as set forth in SEQ ID NO:9 and a light chain variable region amino acid sequence as set forth in SEQ ID NO:10.

In another embodiment, the anti-PD-1 binding domain of the bispecific construct comprises a heavy chain variable region amino acid sequence as set forth in SEQ ID NO:59 and a light chain variable region amino acid sequence as set forth in SEQ ID NO:60. Alternatively, the anti-PD-1 binding domain of the bispecific construct comprises a heavy chain variable region amino acid sequence as set forth in SEQ ID NO:61 and a light chain variable region amino acid sequence as set forth in SEQ ID NO:62.

In another embodiment, the anti-ILT4 binding domain of the bispecific construct comprises a heavy chain variable region amino acid sequence as set forth in SEQ ID NO:19and a light chain variable region amino acid sequence as set forth in SEQ ID NO:20; and the anti-PD-1 binding domain comprises a heavy chain variable region amino acid sequence as set forth in SEQ ID NO:59 and a light chain variable region amino acid sequence as set forth in SEQ ID NO:60.

The bispecific construct can be a chemical conjugate, which can be made by chemical conjugation of the anti-ILT4 binding domain and the anti-PD-1 binding domain. In one embodiment, the anti-PD-1 binding domain further comprises a human IgGI constant domain. In another embodiment, the anti-ILT4 binding domain is linked to the C-terminus of the heavy chain of the anti-PD-1 binding domain. In another embodiment, the anti-ILT4 binding domain is a scFv.

In a particular embodiment, the bispecific construct comprises an anti-PD-1 binding domain linked to an anti-ILT4 scFv, wherein:
(a) the anti-PD-1 binding domain comprises heavy chain variable region CDR1, CDR2 and CDR3 amino acid sequences as set forth in SEQ ID NOs: 31, 36, and 41, respectively, and light chain variable region CDR1, CDR2 and CDR3 amino acid sequences as set forth in SEQ ID NOs: 46, 51, and 56, respectively, and an IgGI constant domain; and
(b) the anti-ILT4 scFv comprises heavy chain variable region CDR1, CDR2 and CDR3 amino acid sequences as set forth in SEQ ID NOs: 11, 13, and 15, respectively, and light chain variable region CDR1, CDR2 and CDR3 amino acid sequences as set forth in SEQ ID NOs: 16, 17, and 18, respectively.

For example, the bispecific construct comprises heavy and light chain sequences as set forth in SEQ ID NOs: 64 and 63, respectively, or encoded by the nucleotide sequences as set forth in SEQ ID NOs: 66 and 65, respectively.

The present invention also provides compositions comprising any of the bispecific constructs described herein and a pharmaceutically acceptable carrier, as well as kits comprising any of the bispecific constructs described herein and instructions for use.

In a further aspect, isolated nucleic acid molecules encoding the bispecific constructs (or portions thereof) described herein are also provided, as well as expression vectors comprising such nucleic acids and host cells comprising such expression vectors. In another embodiment, a nucleic acid molecule coding for any of the bispecific constructs described herein is provided. In another embodiment, the nucleic acid molecule is in the form of an expression vector. In another embodiment, the nucleic acid molecule is in the form of an expression vector which expresses the anti-ILT4 binding domain, the anti-PD-1 binding domain, or both binding domains when administered to a subject in vivo. In another embodiment, the nucleic acid molecule is in the form of an expression vector which expresses the heavy chain, the light chain, or both the heavy and light chains of the bispecific construct when administered to a subject in vivo.

For example, the nucleic acid molecule comprises a nucleotide sequence encoding the heavy chain comprising the amino acid sequence as set forth in SEQ ID NO:64, the light chain comprising the amino acid sequence as set forth in SEQ ID NO:63, both heavy and light chains as set forth in SEQ ID NOs: 64 and 63, or an amino acid sequence at least 90% identical thereto (e.g., at least 90%, 91%, 92%, 93%, 94%, 95%, 96%, 97%, 98% or 99% identical to one or more of the aforementioned sequences).

In another embodiment, the nucleic acid molecule comprises a nucleotide sequence as set forth in SEQ ID NO:66 encoding the heavy chain, a nucleotide sequence as set forth in SEQ ID NO:65 encoding the light chain, a nucleotide sequence encoding both heavy and light chains, or a nucleotide sequence at least 90% identical thereto (e.g., at least 90%, 91%, 92%, 93%, 94%, 95%, 96%, 97%, 98% or 99% identical to one or more of the aforementioned sequences).

In another embodiment, methods for inducing or enhancing an immune response (e.g., against an antigen) in a subject comprising administering to the subject any one of the bispecific constructs or the compositions described herein, in an amount effective to induce or enhance an immune response in the subject (e.g., against an antigen).

In a further embodiment, methods of for treating a condition or disease in a subject (e.g., cancer) are provided, the method comprising administering to the subject any one of the bispecific constructs or the compositions described herein, in an amount effective to treat the condition or disease.

In another embodiment, methods for treating a tumor (e.g., a tumor expressing ILT4, HLA-G, HLA class I, angiopoietin like 2, Nogo, or an ILT4 ligand) in a subject are provided, the method comprising administering to the subject any one of the bispecific constructs or compositions described herein, in an amount effective to treat the tumor.

The subject can be, for example, one who suffers from a condition or disease in which stimulation of an immune response is desired. In one embodiment, the condition or disease in which stimulation of an immune response is desired is cancer. Such methods include administration of one or more therapeutic agents to the subject, e.g., wherein the therapeutic agent is another antibody, such as an anti-CD40 antibody, anti-PD-L1 and/or an anti-CTLA-4 antibody. The bispecific construct (or composition thereof) and the one or more therapeutic agents can be administered concurrently, sequentially.

The method of inducing or enhancing an immune response (e.g., against an antigen) in a subject can further comprise administering the antigen to the subject. Preferred antigens to be co-administered with the bispecific constructs or the compositions of described herein are tumor antigens.

### III. BRIEF DESCRIPTION OF THE DRAWINGS

FIG. 1 shows an image of an SDS gel electrophoresis gel of the bispecific construct, CDX-585, compared to IgG.

FIG. 2 shows a HPLC trace of the bispecific construct, CDX-585, compared to IgG1.

FIG. 3 shows a schematic representation of a bispecific construct according to the invention.

FIG. 4 is a graph showing a representative binding curve of the bispecific construct, CDX-585, to human PD-1 using ELISA.

FIGS. 5A and 5B are graphs showing representative binding curves of the bispecific construct, CDX-585, to cells expressing human PD-1 (FIG. 2A) and cells expressing human ILT4 (FIG. 2B).

FIGS. 6A and 6B are graphs showing binding curves of the bispecific construct, CDX-585, to cell-expressed human ILT4 (FIG. 3A) and human PD-1 (FIG. 3B).

FIG. 7 are tables showing the affinity and rate constants of the bispecific construct, CDX-585 by Bio-Layer Interferometry (BLI).

FIG. 8 is a graph showing blockade of the PD1/PD-L1 interaction by the bispecific construct, CDX-585.

FIGS. 9A and 9B are graphs showing the induction of TNF-α production with the bispecific construct, CDX-585, in dendritic cells (FIG. 6A) and macrophages (FIG. 6B).

FIG. 10 is a graph showing a representative blocking curve of HLA-G binding to ILT4 by the bispecific construct, CDX-585.

FIG. 11 is a graph showing downregulation of PD-L1 expression by the bispecific construct, CDX-585.

FIGS. 12A and 12B are graphs showing the increase in TNF-a production (FIG. 9A) and the downregulation IL-10 secretion (FIG. 9B) by the bispecific construct, CDX-585.

FIGS. 13A and 13B are graphs showing the bispecific construct, CDX-585, induced mixed lymphocyte response as measured by IFN-γ (FIG. 10A) production and mixed lymphocyte response as measured by IL-2 (FIG. 10B) production.

FIG. 14 is a graph showing increase of IFN-γ production with the bispecific construct, CDX-585, indicating a synergistic effect of the combination of anti-ILT4 and anti-PD-1 binding domains.

FIG. 15 is a graph showing in vivo antitumor activity in mouse tumor model.

FIGS. 16A-16D are graphs showing in vivo antitumor activity in mouse tumor model with (A) human IgG1 AQQ (0.5 mg/mouse), (B) 7B1 (0.375 mg/mouse), (C) E1A9 (0.375 mg/mouse) and 7B1 (0.375 mg/mouse), and (D) CDX-585 (0.5 mg/mouse).

FIGS. 17A-17C are graphs showing serum concentrations of cytokine/chemokines in cynomolgus macaques given single a i.v. dose (10 mg/kg) of CDX-585 (A) MCP-1 (CCL2), (B) MIP-1B (CCL4), and (C) MDC (CCL22).

FIG. 18 is a graph showing serum concentrations of CDX-585 in cynomolgus macaques given single a i.v. dose (10 mg/kg) of CDX-585.

FIG. 19 is a graph showing IFN-γ production of antibodies 7B1, E1A9, a combination of 7B1 and E1A9, and CDX-585 in dendritic cells (DCs) incubated with LPS or anti-CD40 antibody (CDX-1140).

FIG. 20 is a graph showing in vivo antitumor activity in mouse tumor model.

### IV. DETAILED DESCRIPTION OF THE INVENTION

In order that the present invention may be more readily understood, certain terms are first defined. Additional definitions are set forth throughout the detailed description.

### Definitions

The term “immunoglobulin-like transcript 4” or “ILT4” as used herein refers to an inhibitory immune checkpoint receptor and a member of the non-catalytic tyrosine-phosphorylated receptor family. ILT4 is also referred to as leukocyte immunoglobulin like receptor B2 (LILRB2), LIR2, MIR10, and CD85d. ILT4 is expressed on immune cells where it binds to MHC class I molecules on antigen-presenting cells and transduces a negative signal that inhibits stimulation of an immune response, e.g., by controlling inflammatory responses and cytotoxicity to focus the immune response and limit autoreactivity. Multiple isoforms of human ILT4 have been identified. Isoform 1 (Accession No. Q8N423-1) represents the canonical sequence, consisting of 598 amino acid residues. Anti-ILT4 binding domains (or portions thereof) of the invention may cross-react with ILT4 from species other than human. Alternatively, the anti-ILT4 binding domains may be specific for human ILT4 and may not exhibit any cross-reactivity with other species. ILT4 or any variants and isoforms thereof, may either be isolated from cells or tissues which naturally express them or be recombinantly produced using well-known techniques in the art and/or those described herein.

Ligands which bind ILT4 are known in the art and include, among others, HLA-G, HLA class I, angiopoietin like 2, b-amyloid, SEMA4A, CD1c/d, CSPs, and myelin inhibitors such as Nogo66, MAG, OMgp.

The terms “human leukocyte antigen G” or “HLA-G” (also known as “histocompatibility antigen, class I, G”), refers to a ligand for ILT4. HLA-G belongs to the HLA nonclassical class I heavy chain paralogues. This class I molecule is a heterodimer consisting of a heavy chain and a light chain (beta-2 microglobulin). The heavy chain is anchored in the membrane. HLA-G is expressed on fetal derived placental cells. The heavy chain is approximately 45 kDa and its gene contains 8 exons.

As used herein, the terms “Programmed Death 1,” “Programmed Cell Death 1,” “Protein PD-1,” “PD-1,” PD1,” “PDCD1,” “hPD-1” and “hPD-I” are used interchangeably, and include variants, isoforms, species homologs of human PD-1, and analogs having at least one common epitope with PD-1. The complete PD-1 sequence can be found under GenBank Accession No. NP_005009.

As used herein, the terms “Programmed cell death 1 ligand 1”, “PD-L1”, “PDCD1 ligand 1”, “Programmed death ligand 1”, “B7 homolog 1”, “B7-H1” and “ILT44” are used interchangeably, and include variants, isoforms, species homologs of human PD-L1, and analogs having at least one common epitope with PD-L1. The complete PD-L1 sequence can be found under GenBank Accession No. NP_001254635. The binding of PD-L1 to PD-1 transmits an inhibitory signal that reduces the proliferation of these T cells and can also induce apoptosis, which is further mediated by a lower regulation of the gene Bcl-2.

As used herein, the term “subject” includes any human or non-human animal. For example, the methods and compositions of the present invention can be used to treat a subject with an immune disorder. The term “non-human animal” includes all vertebrates, e.g., mammals and non-mammals, such as non-human primates, sheep, dog, cow, chickens, amphibians, reptiles, etc.

The term “antibody” as referred to herein refers to a protein comprising at least two heavy (H) chains and two light (L) chains inter-connected by disulfide bonds, or an antigen binding fragment thereof. Each heavy chain may be comprised of a heavy chain variable region (abbreviated herein as VH) and a heavy chain constant region. The heavy chain constant region may be comprised of three domains, CH1, CH2 and CH3. Each light chain may be comprised of a light chain variable region (abbreviated herein as V_{L}) and a light chain constant region. The light chain constant region may be comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} may be composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen binding fragment” of an antibody (or simply “antibody fragment”), as used herein, refers to one or more fragments or portions of an antibody that retain the ability to specifically bind to an antigen (e.g., human ILT4). Such “fragments” are, for example between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen binding function of an antibody can be performed by fragments of a full-length antibody. Examples of binding fragments encompassed within the term “antigen binding fragment” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and

V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (sFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen binding fragment” of an antibody. These antibody fragments are obtained using conventional techniques known to those with skill in the art, and the fragments are screened for utility in the same manner as are intact antibodies. Antigen binding fragments can be produced by recombinant DNA techniques, or by enzymatic or chemical cleavage of intact immunoglobulins.

As used herein, the term “binding domain” refers to the portion of a protein or antibody which comprises the amino acid residues that interact with an antigen. Binding domains include, but are not limited to, antibodies (e.g., full length antibodies), as well as antigen-binding portions thereof. The binding domain confers on the binding agent its specificity and affinity for the antigen. The term also covers any protein having a binding domain which is homologous or largely homologous to an immunoglobulin-binding domain. Such proteins may be derived from natural sources, or partly or wholly synthetically produced.

As used herein, the terms “bispecific construct,” “bispecific antibody,” and “bsAb,” refer to constructs and antibodies having linked binding domains that can bind to two different antigens at the same time. Bispecific constructs that have affinities for two different epitopes bind to two targets, either monovalently or bivalently depending on the construct. Bispecific constructs can be produced according to a variety of methods, such as conjugating two existing binding domains, fusing two hybridoma cell lines to form a quadroma (Jain et al.

(2007) Trends in Biotechnology 25(7), 307-316), or using genetically engineered recombinant proteins (Kontermann (2012) Dual targeting strategies with bispecific antibodies. mAbs 4(2), 182-197).

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

The term “monoclonal antibody,” as used herein, refers to an antibody that displays a single binding specificity and affinity for a particular epitope. Accordingly, the term “human monoclonal antibody” refers to an antibody which displays a single binding specificity and which has variable and optional constant regions derived from human germline immunoglobulin sequences. In one embodiment, human monoclonal antibodies are produced by a hybridoma that includes a B cell obtained from a transgenic non-human animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg (2005) Nature Biotech. 23(9): 1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen may not have sequence identity with the original nucleic acid molecules, but instead will be substantially identical or similar (i.e., have at least 80% identity).

The term “human antibody” includes antibodies having variable and constant regions (if present) of human germline immunoglobulin sequences. Human antibodies of the invention can include amino acid residues not encoded by human germline immunoglobulin sequences (e.g., mutations introduced by random or site-specific mutagenesis in vitro or by somatic mutation in vivo) (see, Lonberg, N. et al. (1994) Nature 368 (6474): 856-859); Lonberg, N. (1994) Handbook of Experimental Pharmacology 113:49-101; Lonberg, N. and Huszar, D. (1995) Intern. Rev. Immunol. Vol. 13:65-93, and Harding, F. and Lonberg, N. (1995) Ann. N.Y. Acad. Sci 764:536-546). However, the term “human antibody” does not include antibodies in which CDR sequences derived from the germline of another mammalian species, such as a mouse, have been grafted onto human framework sequences (i.e., chimeric and humanized antibodies).

A “humanized” antibody refers to an antibody in which some, most, or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

An “isolated antibody,” as used herein, is intended to refer to an antibody which is substantially free of other antibodies having different antigenic specificities (e.g., an isolated antibody that specifically binds to human ILT4 is substantially free of antibodies that specifically bind antigens other than human ILT4; an isolated antibody that specifically binds to human PD-1 is substantially free of antibodies that specifically bind antigens other than human PD-1). An isolated antibody that specifically binds to an epitope may, however, have cross-reactivity to the same antigen from different species. In addition, an isolated antibody is typically substantially free of other cellular material and/or chemicals.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from the antigen (e.g., ILT4 or PD-1) are tested for reactivity with the given antibody (e.g., an ILT4 or PD-1 antibody). Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

The term “antibody that binds the same epitope” as another antibody is intended to encompass antibodies that interact with, i.e., bind to, the same structural region on human ILT4 as a reference ILT4 antibody. The “same epitope” to which the antibodies bind may be a linear epitope or a conformational epitope formed by tertiary folding of the antigen.

The term “competing antibody” refers to an antibody that competes for binding to human ILT4 with a reference ILT4 antibody, i.e., competitively inhibits binding of the reference ILT4 antibody to ILT4. A “competing antibody” may bind the same epitope on ILT4 as the reference ILT4 antibody, may bind to an overlapping epitope or may sterically hinder the binding of the reference ILT4 antibody to ILT4.

Antibodies that recognize the same epitope or compete for binding can be identified using routine techniques. Such techniques include, for example, an immunoassay, which shows the ability of one antibody to block the binding of another antibody to a target antigen, i.e., a competitive binding assay. Competitive binding is determined in an assay in which the immunoglobulin under test inhibits specific binding of a reference antibody to a common antigen, such as ILT4. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25 (1): 7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

Other techniques include, for example, epitope mapping methods, such as, x-ray analyses of crystals of antigen: antibody complexes which provides atomic resolution of the epitope. Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. The peptides are then regarded as leads for the definition of the epitope corresponding to the antibody used to screen the peptide library. For epitope mapping, computational algorithms have also been developed which have been shown to map conformational discontinuous epitopes.

As used herein, the terms “specific binding,” “selective binding,” “selectively binds,” and “specifically binds,” refer to antibody binding to an epitope on a predetermined antigen. Typically, the antibody binds with an equilibrium dissociation constant (KD) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE 2000 instrument (e.g., using recombinant human ILT4 as the analyte and the antibody as the ligand) and binds to the predetermined antigen with an affinity that is at least two-fold greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.”

The term “K_{D},” as used herein, is intended to refer to the dissociation equilibrium constant of a particular antibody-antigen interaction. Typically, the human antibodies of the invention bind to ILT4 with a dissociation equilibrium constant (K_{D}) of approximately 10⁻⁸ M or less, such as less than 10⁻⁹ M or 10⁻¹⁰ M or even lower when determined by surface plasmon resonance (SPR) technology in a BIACORE 2000 instrument (e.g., using recombinant human ILT4 as the analyte and the antibody as the ligand).

The term “kd” as used herein, is intended to refer to the off rate constant for the dissociation of an antibody from the antibody/antigen complex.

The term “ka” as used herein, is intended to refer to the on rate constant for the association of an antibody with the antigen.

The term “EC50,” as used herein, refers to the concentration of an antibody or an antigen binding fragment thereof, which induces a response, either in an in vitro or an in vivo assay, which is 50% of the maximal response, i.e., halfway between the maximal response and the baseline.

As used herein, “isotype” refers to the antibody class (e.g., IgM or IgGI) that is encoded by heavy chain constant region genes. In one embodiment, a human monoclonal antibody of the invention is of the IgGI isotype. In another embodiment, a human monoclonal antibody of the invention is of the IgG2 isotype.

As used herein, the terms “inhibits” or “blocks” (e.g., referring to inhibition/blocking of binding of HLA-G ligand to ILT4 and/or PD1 to PD-L1 ligand) are used interchangeably and encompass both partial and complete inhibition/blocking. The inhibition/blocking preferably reduces or alters the normal level or type of activity that occurs when binding occurs without inhibition or blocking. Inhibition and blocking are also intended to include any measurable decrease in the binding affinity of HLA-G when in contact with an anti-ILT4 binding domain, antibody, or portion thereof, as compared to HLA-G not in contact with an ILT4 binding domain, antibody, or portion thereof, e.g., inhibits binding of HLA-G by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In one embodiment, the anti-ILT4 binding domain inhibits binding of HLA-G by at least about 70%. In another embodiment, the anti-ILT4 binding domain inhibits binding of HLA-G by at least 80%. Inhibition and blocking are also intended to include any measurable decrease in the binding affinity of PD-1 when in contact with an PD-L1 binding domain, antibody, or portion thereof as compared to PD-1 not in contact with an PD-L1 binding domain, antibody, or portion thereof, e.g., inhibits binding of PD-1 by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In one embodiment, the PD-1 binding domain inhibits binding of PD1 by at least about 70%. In another embodiment, the PD-1 binding domain inhibits binding of PD1 by at least 80%.

The term “cross-reacts,” as used herein, refers to the ability of an anti-ILT4 binding domain, antibody, or portion thereof, or an anti-PD-1 binding domain, antibody, or portion thereof, of the invention to bind to ILT4 or PD-1, respectively, from a different species. For example, an anti-ILT4 binding domain of the invention that binds human ILT4 may also bind another species of ILT4. Similarly, an anti-PD-1 binding domain of the invention that binds human PD-1 may also bind another species of PD-1. As used herein, cross-reactivity is measured by detecting a specific reactivity with purified antigen in binding assays (e.g., SPR, ELISA) or binding to, or otherwise functionally interacting with, cells physiologically expressing ILT4. Methods for determining cross-reactivity include standard binding assays as described herein, for example, by Biacore™ surface plasmon resonance (SPR) analysis using a Biacore™ 2000 SPR instrument (Biacore AB, Uppsala, Sweden), or flow cytometric techniques.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “nucleic acid molecule,” as used herein, is intended to include DNA molecules and RNA molecules. A nucleic acid molecule may be single-stranded or double-stranded, but preferably is double-stranded DNA.

The term “isolated nucleic acid molecule,” as used herein in reference to nucleic acids encoding binding domains, antibodies, or portions thereof (e.g., V_{H}, V_{L}, CDR3) that bind to ILT4 and/or PD-1, is intended to refer to a nucleic acid molecule in which the nucleotide sequences encoding the binding domains, antibodies, or portions are free of other nucleotide sequences encoding the binding domains, antibodies, or portions that bind antigens other than ILT4 and/or PD-1, which other sequences may naturally flank the nucleic acid in human genomic DNA.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

The nucleic acid molecules of the present invention, while often in a native sequence (except for modified restriction sites and the like), from either cDNA, genomic or mixtures thereof may be mutated, in accordance with standard techniques to provide gene sequences. For coding sequences, these mutations, may affect amino acid sequence as desired. In particular, DNA sequences substantially identical to or derived from native V, D, J, constant, switches and other such sequences described herein are contemplated (where “derived” indicates that a sequence is identical or modified from another sequence).

A nucleic acid is “operably linked” or “operatively linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The present invention also encompasses “conservative sequence modifications” of any of the sequences described herein, i.e., nucleotide and amino acid sequence modifications which do not abrogate the binding of the VH and VL sequences encoded by the nucleotide sequence or containing the amino acid sequence, to the antigen. Such conservative sequence modifications include conservative nucleotide and amino acid substitutions, as well as, nucleotide and amino acid additions and deletions. For example, modifications can be introduced into the sequences by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-ILT4 binding domain is preferably replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10): 879-884 (1999); and Burks et al. Proc. Natl. Acad. Sci. USA 94:412-417 (1997)). In certain embodiments, conservative amino acid sequence modifications refer to at most 1, 2, 3, 4 or 5 conservative amino acid substitutions to the CDR sequences described herein. For example, each such CDR may contain up to 5 conservative amino acid substitutions, e.g., up to (i.e., not more than) 4 conservative amino acid substitutions, e.g.,up to (i.e., not more than) 3 conservative amino acid substitutions, e.g., up to (i.e., not more than) 2 conservative amino acid substitutions, or no more than 1 conservative amino acid substitution.

Alternatively, in another embodiment, mutations can be introduced randomly along all or part of a coding sequence for an anti-ILT4 or anti-PD-1 binding domain, antibody, or portion thereof, such as by saturation mutagenesis, and the resulting modified anti-ILT4 or anti-PD-1 binding domain, antibody, or portion can be screened for binding activity.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

For amino acids, the term “substantial homology” indicates that two amino acid sequences, or designated sequences thereof, when optimally aligned and compared, are identical, with appropriate amino acid insertions or deletions, in at least about 80% of the amino acids, usually at least about 90% to 95%, and more preferably at least about 98% to 99% or 99.5% of the amino acids.

The percent identity between two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present invention can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences identical to the nucleic acid molecules of the invention. BLAST protein searches can be performed with the XBLAST program, score=50, wordlength=3 to obtain amino acid sequences identical to the protein molecules of the invention. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used. See http://www.ncbi.nlm.nih.gov.

### ILT4 Binding Domains

Provided herein are novel bispecific constructs comprising anti-ILT4 binding domains, for example, binding domains derived from antibodies, e.g., humanized antibodies, which are characterized by particular functional features or properties. For example, such binding domains of the present invention exhibit one or more of the following properties:
a. blocking ILT4 ligand (e.g., HLA-G ligand) binding to human ILT4;
b. enhancing or increasing cytokine or chemokine release by human macrophages;
c. potentiating the activation effects of LPS and IFNγ on macrophages;
d. promoting Ml macrophage polarization;
e. binding to human ILT4 with an equilibrium dissociation constant Kd of 10⁻⁹ M or less, or alternatively, an equilibrium association constant Ka of 10⁺⁹ M⁻¹ or greater;
f. lack of cross-reactivity with other ILT family members;
g. cross-reactivity with cynomolgus ILT4; and/or
h. inhibiting tumor cells that express ILT4.

In one embodiment, the anti-ILT4 binding domain is derived from antibody 7A3 as described herein. For example, the anti-ILT4 binding domain comprises the heavy and light chain CDRs or variable regions of antibody 7A3. In another embodiment, the binding domain comprises the CDR1, CDR2, and CDR3 domains of the heavy chain variable region of antibody 7A3 having the sequence set forth in SEQ ID NO:9, and the CDR1, CDR2 and CDR3 domains of the light chain variable region of antibody 7A3 having the sequence set forth in SEQ ID NO:10. In another embodiment, the binding domain comprises heavy chain CDR1, CDR2 and CDR3 domains having the sequences set forth in SEQ ID NOs: 1, 3, and 5, respectively, or conservative sequence modifications thereof, and light chain CDR1, CDR2and CDR3 domains having the sequences set forth in SEQ ID NOs: 6, 7, and 8, respectively, or conservative sequence modifications thereof. Alternatively, the binding domain comprises heavy chain CDR1, CDR2 and CDR3 domains having the sequences set forth in SEQ ID NOs: 2, 4, and 5, respectively, or conservative sequence modifications thereof, and light chain CDR1, CDR2 and CDR3 domains having the sequences set forth in SEQ ID NOs: 6, 7, and 8, respectively, or conservative sequence modifications thereof. In another embodiment, the binding domain comprises a heavy chain variable region having the amino acid sequence set forth in SEQ ID NO:9. In another embodiment, the binding domain comprises a light chain variable region having the amino acid sequence set forth in SEQ ID NO:10. For example, the anti-ILT4 binding domain comprises heavy and light chain variable regions having the amino acid sequences set forth in SEQ ID NO:9 and 10.

Another exemplary anti-ILT4 binding domain is derived from antibody 7B1 as described herein. In one embodiment, the anti-ILT4 binding domain comprises the heavy and light chain CDRs or variable regions of antibody 7B1. In another embodiment, the binding domain comprises the CDR1, CDR2, and CDR3 domains of the heavy chain variable region of antibody 7B1 having the sequence set forth in SEQ ID NO:19, and the CDR1, CDR2 and CDR3 domains of the light chain variable region of antibody 7B1 having the sequence set forth in SEQ ID NO:20. In another embodiment, the binding domain comprises heavy chain CDR1, CDR2 and CDR3 domains having the sequences set forth in SEQ ID NOs: 11, 13, and 15, respectively, or conservative sequence modifications thereof, and light chain CDR1, CDR2 and CDR3 domains having the sequences set forth in SEQ ID NOs: 16, 17, and 18, respectively, or conservative sequence modifications thereof. Alternatively, the binding domain comprises heavy chain CDR1, CDR2 and CDR3 domains having the sequences set forth in SEQ ID NOs: 12, 14, and 15, respectively, or conservative sequence modifications thereof, and light chain CDR1, CDR2 and CDR3 domains having the sequences set forth in SEQ ID NOs: 16, 17, and 18, respectively, or conservative sequence modifications thereof. In another embodiment, the binding domain comprises a heavy chain variable region having the amino acid sequence set forth in SEQ ID NO:19. In another embodiment, the binding domain comprises a light chain variable region having the amino acid sequence set forth in SEQ ID NO:20. For example, the binding domain comprises heavy and light chain variable regions having the amino acid sequences set forth in SEQ ID NO:19 and 20.

The anti-ILT4 binding domain can also be consensus sequences of antibodies 7B1 and 7A3. For example, in one embodiment, the anti-ILT4 binding domain comprises a heavy chain variable region CDRI comprising an amino acid sequence selected from the consensus sequence: G Y T (I,M) H (SEQ ID NO: 21). In another embodiment, the anti-ILT4 binding domain comprises a heavy chain variable region CDR2 comprising SEQ ID NO:3. In another embodiment, the anti-ILT4 binding domain comprises a heavy chain variable region CDR3 comprising an amino acid sequence selected from the consensus sequence: E R P G G S Q F I Y Y Y (P,A) (M,L) D Y (SEQ ID NO:22). In another embodiment, the anti-ILT4binding domain comprises a light chain variable region CDRI comprising an amino acid sequence selected from the consensus sequence: R A S (A,E) NI Y S Y LA (SEQ ID NO: 23). In another embodiment, the anti-ILT4 binding domain comprises a light chain variable region CDR2 comprising an amino acid sequence selected from the consensus sequence: N A (I,D) T L A E (SEQ ID NO: 24). In another embodiment, the anti-ILT4 binding domain comprises a light chain variable region CDR3 comprising SEQ ID NO:8.

Given that each of the described antibodies can bind to human ILT4, the V_{H} and V_{L} sequences described herein can be “mixed and matched” to create various anti-ILT4 binding domains. The binding of such “mixed and matched” binding domains to human ILT4 can be tested using the binding assays known in the art and described in the Examples (e.g., ELISAs). For example, anti-ILT4 binding domains of the present invention include combinations of the 7B1 and 7A3 antibody heavy and light chain variable region sequences described herein.

Sequences substantially identical to the anti-ILT4 binding domains described herein (e.g., sequences at least 80%, 81%, 82%, 83%, 84%, 85%, 86%, 87%, 88%, 89%, 90%, 91%, 92%, 93%, 94%, 95%, 96%, 97%, 98% or 99% identical to the aforementioned sequences) are also provided. For example, in one embodiment, the anti-ILT4 binding domain comprises a heavy chain variable region comprising SEQ ID NO:9, 19, or a sequence at least 80% identical thereto (e.g., at least 80%, 81%, 82%, 83%, 84%, 85%, 86%, 87%, 88%, 89%, 90%, 91%, 92%, 93%, 94%, 95% 96%, 97%, 98%, or 99% identical to the aforementioned sequences). In another embodiment, the anti-ILT4 binding domain comprises a light chain variable region comprising SEQ ID NO:10, 20, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical to the aforementioned sequences). In another embodiment, the anti-ILT4 binding domain comprises a (a) heavy chain variable region comprising SEQ ID NO:9, 19, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical to the aforementioned sequences) and (b) light chain variable region comprising SEQ ID NO: 10, 20, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical to the aforementioned sequences). For example, the anti-ILT4 binding domain comprises SEQ ID NO: 9, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical thereto) and SEQ ID NO:19, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical thereto). Alternatively, the anti-ILT4 binding domain comprises SEQ ID NO:10, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical thereto) and SEQ ID NO:20, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical thereto).

In one embodiment, the anti-ILT4 binding domain comprises heavy chain variable region CDR1, CDR2 and CDR3 as set forth in SEQ ID NOs: 1, 3, and 5, respectively, and light chain variable region CDR1, CDR2 and CDR3 as set forth in SEQ ID NOs: 6, 7, and 8, respectively. Alternatively, the anti-ILT4 binding domain comprises heavy chain variable region CDR1, CDR2 and CDR3 as set forth in SEQ ID NOs: 2, 4, and 5, respectively, and light chain variable region CDR1, CDR2 and CDR3 as set forth in SEQ ID NOs: 6, 7, and 8, respectively. In another embodiment, the anti-ILT4 binding domain comprises a heavy chain variable region comprising SEQ ID NO:9 and a light chain variable region comprising SEQ ID NO: 19 or sequences at least 80% identical to the aforementioned sequences (e.g., at least 80%, 81%, 82%, 83%, 84%, 85%, 86%, 87%, 88%, 89%, 90%, 91%, 92%, 93%, 94%, 95%, 96%, 97%, 98% or 99% identical).

In another embodiment, the anti-ILT4 binding domain comprises heavy chain variable region CDR1, CDR2 and CDR3 as set forth in SEQ ID NOs: 11, 13, and 15, respectively, and light chain variable region CDR1, CDR2 and CDR3 as set forth in SEQ ID NOs: 16, 17, and 18, respectively. Alternatively, the anti-ILT4 binding domain comprises heavy chain variable region CDR1, CDR2 and CDR3 as set forth in SEQ ID NOs: 12, 14, and 15, respectively, and light chain variable region CDR1, CDR2 and CDR3 as set forth in SEQ ID NOs: 16, 17, and 18, respectively. In another embodiment, the anti-ILT4 binding domain comprises a heavy chain variable region comprising SEQ ID NO:10 and a light chain variable region comprising SEQ ID NO:20 or sequences at least 80% identical to the aforementioned sequences (e.g., at least 80%, 81%, 82%, 83%, 84%, 85%, 86%, 87%, 88%, 89%, 90%, 91%, 92%, 93%, 94%, 95%, 96%, 97%, 98% or 99% identical).

### PD-1 Binding Domains

Provided herein are anti-PD-1 binding domains thereof for use with the anti-ILT4 binding domains of the present invention, e.g., in bispecific constructs, as well as methods of treatment. Such anti-PD-1 binding domains are derived from antibodies, e.g., humanized antibodies. Exemplary PD-1 antibodies include antibody ElA9C8A7-V8-3 (also referred to a antibody “ElA9” herein) and antibody ElA9C8A7-V8, as described herein.

In one embodiment, the anti-PD-1 binding domain comprises the heavy and light chain CDRs or variable regions of antibody ElA9. In another embodiment, the binding domain comprises the CDR1, CDR2, and CDR3 domains of the heavy chain variable region of antibody ElA9 having the sequence set forth in SEQ ID NO:59 or 61, and the CDR1, CDR2 and CDR3 domains of the light chain variable region of antibody ElA9 having the sequence set forth in SEQ ID NO: 60 or 62. In another embodiment, the binding domain comprises heavy chain CDR1, CDR2 and CDR3 domains having the sequences set forth in SEQ ID NOs: 31, 36, and 41, respectively, or conservative sequence modifications thereof, and light chain CDR1, CDR2 and CDR3 domains having the sequences set forth in SEQ ID NOs: 46, 51, and 56, respectively, or conservative sequence modifications thereof. Alternatively, the binding domain comprises heavy chain CDR1, CDR2 and CDR3 domains having the sequences set forth in SEQ ID NOs: 69, 74, and 79, respectively, or conservative sequence modifications thereof, and light chain CDR1, CDR2 and CDR3 domains having the sequences set forth in SEQ ID NOs: 84, 89, and 94, respectively, or conservative sequence modifications thereof.

In another embodiment, the binding domain comprises a heavy chain variable region having the amino acid sequence set forth in SEQ ID NO:59 or SEQ ID NO:61. In another embodiment, the binding domain comprises a light chain variable region having the amino acid sequence set forth in SEQ ID NO:60 or SEQ ID NO:62. In another embodiment, the binding domain comprises heavy and light chain variable regions having the amino acid sequences set forth in SEQ ID NO:59 and SEQ ID NO:60, respectively. Alternatively, the binding domain comprises heavy and light chain variable regions having the amino acid sequences set forth in SEQ ID NO:61 and SEQ ID NO:62, respectively.

Sequences substantially identical to the anti-PD-1 binding domains described herein (e.g., at least 80%, 85%, 90%, 91%, 92%, 93%, 94%, 95%, 96%, 97%, 98% or 99% identical to the aforementioned sequences), are also encompassed by the invention. In one embodiment, the anti-PD-1 binding domain comprises a heavy chain variable region comprising SEQ ID NO:59, SEQ ID NO:61, or a sequence at least 90% identical thereto (e.g., at least 90%, 91%, 92%, 93%, 94%, 95%, 96%, 97%, 98% or 99% identical to the aforementioned sequences). In another embodiment, the anti-PD-1 binding domain comprises a light chain variable region comprising SEQ ID NO:60, SEQ ID NO:62, or a sequence at least 90% identical thereto (e.g., at least 90%, 91%, 92%, 93%, 94%, 95%, 96%, 97%, 98% or 99% identical to the aforementioned sequences). In another embodiment, the anti-PD-1 binding domain comprises a heavy chain variable region comprising SEQ ID NO: 59 and a light chain variable region comprising SEQ ID NO:60 or sequences at least 90% identical thereto (e.g., at least 90%, 91%, 92%, 93%, 94%, 95%, 96%, 97%, 98% or 99% identical to the aforementioned sequences).

In another embodiment, the anti-PD-1 binding domain comprises heavy chain variable region CDR1, CDR2 and CDR3 as set forth in SEQ ID NOs: 31, 36, and 41, respectively, and light chain variable region CDR1, CDR2 and CDR3 as set forth in SEQ ID NOs: 46, 51, and 56, respectively. In another embodiment, the anti-PD-1 binding domain comprises a heavy chain variable region comprising SEQ ID NO:59 and a light chain variable region comprising SEQ ID NO:60 or sequences at least 90% identical thereto (e.g., at least 90%, 91%, 92%, 93%, 94%, 95%, 96%, 97%, 98% or 99% identical to the aforementioned sequences).

In another embodiment, the anti-PD-1 binding domain comprises heavy chain variable region CDR1, CDR2 and CDR3 as set forth in SEQ ID NOs: 69, 74, and 79, respectively, and light chain variable region CDR1, CDR2 and CDR3 as set forth in SEQ ID NOs: 84, 89, and 94, respectively. In another embodiment, the anti-PD-1 binding domain comprises a heavy chain variable region comprising SEQ ID NO:61 and a light chain variable region comprising SEQ ID NO:62 or sequences at least 90% identical thereto (e.g., at least 90%, 91%, 92%, 93%, 94%, 95%, 96%, 97%, 98% or 99% identical to the aforementioned sequences).

In another embodiment, the anti-PD-1 binding domain has one or more of the following functional features: (a) blocks binding of PD-1 to PD-L1 (e.g., partially or completely), (b) induces NFAT pathway activation, and/or (c) induces a mixed lymphocyte reaction.

### Bispecific Constructs

Bispecific constructs can be produced according to a variety of methods, such as conjugating two existing binding domains, fusing two hybridoma cell lines to form a quadroma (Jain et al. (2007) Trends in Biotechnology 25 (7), 307-316), or using genetically engineered recombinant proteins (Kontermann (2012) Dual targeting strategies with bispecific antibodies. mAbs 4 (2), 182-197).

For chemical conjugation, suitable reagents and methods are known in the art for coupling two or more binding domains (e.g., two or more antibodies, or fragments thereof) together. A variety of coupling or crosslinking agents are commercially available and can be used to conjugate the anti-ILT4 binding domain and anti-PD-1 binding domain. Non-limiting examples include Sulfo-SMCC, protein A, carboiimide, dimaleimide, dithio-bis-nitrobenzoic acid (DTNB), and N-succinimidyl-3-(2-pyridyldithio) propionate (SPDP). Sulfo-SMCC, SPDP and DTNB are preferred agents, with Sulfo-SMCC being particularly preferred. Other suitable procedures for crosslinking components (e.g., antibodies or antigen binding fragments thereof) with cross-linking agents are known in the art. See e.g., Karpovsky, B. et al., (1984) J. Exp. Med. 160:1686; Liu, M. A. et al., (1985) Proc. Natl. Acad. Sci USA 82:8648; Segal, D. M. and Perez, P., U.S. Pat. No. 4,676,980; and Brennan, M. (1986) Biotechniques 4:424.

For genetic engineering, nucleic acid molecules encoding the anti-ILT4 binding domain thereof can be inserted into an appropriate expression vector using standard recombinant DNA techniques. A nucleic acid molecule(s) encoding the anti-PD-1 binding domain thereof also can be inserted into the same expression vector, such that it is operatively linked (e.g., in-frame cloning) to the anti-ILT4 binding domain, thereby resulting in an expression vector that encodes a fusion protein that is the bispecific construct. Preferably, the anti-ILT4 binding domain is operatively linked to the C-terminal region of the heavy chain of the anti-PD-1 binding domain. In another embodiment, the anti-PD-1 binding domain is operatively linked to the C-terminal region of the heavy chain of the anti-ILT4 binding domain. Other suitable expression vectors and cloning strategies for preparing the bispecific constructs described herein are known in the art.

For expression of the bispecific constructs in host cells, the coding regions of the binding domains are combined with cloned promoter, leader sequence, translation initiation, leader sequence, constant region, 3′ untranslated, polyadenylation, and transcription termination, sequences to form expression vector constructs. These constructs can be used to express, for example, full length human IgG₁κ or IgG₄κ antibodies. Fully human, humanized and chimeric antibodies used in the bispecific constructs described herein also include IgG2, IgG3, IgE, IgA, IgM, and IgD antibodies. Similar plasmids can be constructed for expression of other heavy chain isotypes, or for expression of antibodies comprising lambda light chains.

Following preparation of an expression vector encoding the bispecific construct, the bispecific construct can be expressed recombinantly in a host cell using standard transfection methods. For example, in one embodiment, nucleic acid encoding the bispecific construct can be ligated into an expression vector, such as a eukaryotic expression plasmid, such as used by GS gene expression system disclosed in WO 87/04462, WO 89/01036 and EP 338 841 or other expression systems well known in the art. The purified plasmid with the cloned bispecific construct gene can be introduced in eukaryotic host cells, such as CHO-cells or NSO-cells or alternatively other eukaryotic cells like a plant derived cells, fungi or yeast cells. The method used to introduce these genes could be methods described in the art, such as electroporation, lipofectin, lipofectamine or other. After introducing the expression vector in the host cells, cells expressing the bispecific construct can be identified and selected. These cells represent the transfectomas that can then be amplified for their expression level and upscaled to produce bispecific constructs. Alternatively, these cloned bispecific constructs can be expressed in other expression systems, such as E. coli or in complete organisms or can be synthetically expressed. Recombinant bispecific constructs can be isolated and purified from these culture supernatants and/or cells.

A bispecific construct of the invention, whether prepared by chemical conjugation or by genetic engineering, can be isolated and purified using one or more methodologies for protein purification well established in the art. Preferred methods for isolation and purification include, but are not limited to, gel filtration chromatography, affinity chromatography, anion-exchange chromatography and the like. A particularly preferred method is gel filtration chromatography, e.g., using a Superdex 200 column. Isolated and purified bispecific constructs can be evaluated using standard methods such as SDS-PAGE analysis.

Accordingly, in one embodiment, the anti-ILT4 binding domain is genetically fused to a anti-PD-1 binding domain. In another embodiment, the anti-ILT4 binding domain and the anti-PD-1 binding domain are chemically conjugated. In one embodiment, the anti-PD-1 binding domain further comprises a human IgGI constant domain. In another embodiment, the anti-ILT4 binding domain is linked to the C-terminus of the heavy chain of the anti-PD-1 binding domain. In another embodiment, the anti-ILT4 binding domain is a scFv. In another embodiment, the anti-ILT4 binding domain further comprises a human IgGI constant domain. In another embodiment, the anti-PD-1 binding domain is linked to the C-terminus of the heavy chain of the anti-ILT4 binding domain. In another embodiment, the anti-PD-1 binding domain is a scFv.

Bispecific constructs comprising sequences substantially identical to the aforementioned anti-ILT4 and anti-PD-1 binding domain sequences (i.e., CDR and variable region sequences) also are provided herein (e.g., sequences having conservative sequence modifications and/or sequences at least 80%, 81%, 82%, 83%, 84%, 85%, 86%, 87%, 88%, 89%, 90%, 91%, 92%, 93%, 94%, 95%, 96%, 97%, 98% or 99% identical to the aforementioned sequences). For example, in one embodiment, the anti-PD-1 binding domain and the anti-ILT4 scFv comprise heavy and light chain sequences as set forth in SEQ ID NOs: 64 and 63, respectively. In another embodiment, the anti-PD-1 binding domain and the anti-ILT4 scFv heavy and light chains are encoded by the nucleotide sequences as set forth in SEQ ID NOs: 66 and 65, respectively.

In another embodiment, the bispecific constructs exhibit one or more of the following properties:
a. blocking ILT4 ligand (e.g., HLA-G ligand) binding to human ILT4;
b. enhancing or increasing cytokine or chemokine release by human macrophages;
c. potentiating the activation effects of LPS and IFNy on macrophages;
d. promoting M1 macrophage polarization;
e. binding to human ILT4 with an equilibrium dissociation constant Kd of 10⁻⁹ M or less, or alternatively, an equilibrium association constant Ka of 10+9 M⁻¹ or greater;
f. lack of cross-reactivity with other ILT family members;
g. cross-reactivity with cynomolgus ILT4;
h. inhibiting tumor cells that express ILT4; and/or
i. enhanced MLR activity compared to the combination of the separate antibodies.

### Compositions

Also provided herein are compositions, e.g., a composition comprising a bispecific construct described herein, formulated together with a carrier (e.g., a pharmaceutically acceptable carrier).

As used herein, the terms “carrier” and “pharmaceutically acceptable carrier” includes any and all solvents, salts, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound (i.e., any of the bispecific constructs or the compositions described herein), may be coated in a material to protect the compound from the action of acids and other natural conditions that may inactivate the compound.

Examples of adjuvants which may be used with the the bispecific constructs and compositions described here include, but are not limited to: Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatised polysaccharides; polyphosphazenes; biodegradable microspheres; cytokines, such as GM-CSF, interleukin-2, -7, -12, and other like factors; 3D-MPL; CpG oligonucleotide; and monophosphoryl lipid A, for example 3-de-O-acylated monophosphoryl lipid A.

MPL adjuvants are available from Corixa Corporation (Seattle, Wash; see, for example, U.S. Pat. Nos. 4,436,727; 4,877,611; 4,866,034 and 4,912,094). CpG-containing oligonucleotides (in which the CpG dinucleotide is unmethylated) are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996.

Further alternative adjuvants include, for example, saponins, such as Quil A, or derivatives thereof, including QS21 and QS7 (Aquila Biopharmaceuticals Inc., Framingham, Mass.); Escin; Digitonin; or Gypsophila or Chenopodium quinoa saponins; Montanide ISA 720 (Seppic, France); SAF (Chiron, California, United States); ISCOMS (CSL), MF-59 (Chiron); the SBAS series of adjuvants (e.g., SBAS-2 or SBAS-4, available from SmithKline Beecham, Rixensart, Belgium); Detox (Enhanzyn™) (Corixa, Hamilton, Mont.); RC-529 (Corixa, Hamilton, Mont.) and other aminoalkyl glucosaminide 4-phosphates (AGPs); polyoxyethylene ether adjuvants such as those described in WO 99/52549A1; synthetic imidazoquinolines such as imiquimod [S-26308, R-837], (Harrison, et al., Vaccine 19:1820-1826, 2001; and resiquimod [S-28463, R-848] (Vasilakos, et al., Cellular immunology 204:64-74, 2000; Schiff bases of carbonyls and amines that are constitutively expressed on antigen presenting cell and T-cell surfaces, such as tucaresol (Rhodes, J. et al., Nature 377:71-75, 1995); cytokine, chemokine and co-stimulatory molecules as either protein or peptide, including for example pro-inflammatory cytokines such as Interferon, GM-CSF, IL-1 alpha, IL-1 beta, TGF-alpha and TGF-beta, Th1 inducers such as interferon gamma, IL-2, IL-12, IL-15, IL-18 and IL-21, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L; immunostimulatory agents targeting ligands such as CTLA-4 and L-selectin, apoptosis stimulating proteins and peptides such as Fas; synthetic lipid based adjuvants, such as vaxfectin, (Reyes et al., Vaccine 19:3778-3786, 2001) squalene, alpha-tocopherol, polysorbate 80, DOPC and cholesterol; endotoxin, [LPS], (Beutler, B., Current Opinion in Microbiology 3:23-30, 2000); ligands that trigger Toll receptors to produce Th1-inducing cytokines, such as synthetic Mycobacterial lipoproteins, Mycobacterial protein p19, peptidoglycan, teichoic acid and lipid A; and CT (cholera toxin, subunits A and B) and LT (heat labile enterotoxin from E. coli, subunits A and B), heat shock protein family (HSPs), and LLO (listeriolysin O; WO 01/72329). These and various further Toll-like Receptor (TLR) agonists are described for example in Kanzler et al, Nature Medicine, May 2007, Vol 13, No 5.

A “pharmaceutically acceptable salt” refers to a salt that retains the desired biological activity of the parent compound and does not impart any undesired toxicological effects (see e.g., Berge, S.M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono-and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A composition of the present invention can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Acceptable diluents include saline and aqueous buffer solutions. Liposomes include water-in-oil-in-water CGF emulsions as well as conventional liposomes (Strejan et al. (1984) J. Neuroimmunol. 7:27).

Carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, the bispecific constructs (and compositions) of the invention may be administered once or twice weekly by subcutaneous or intramuscular injection or once or twice monthly by subcutaneous or intramuscular injection.

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred per cent, this amount will range from about 0.001 per cent to about ninety percent of active ingredient, preferably from about 0.005 per cent to about 70 per cent, most preferably from about 0.01 per cent to about 30 per cent.

Formulations of the present invention which are suitable for vaginal administration also include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

When the compounds of the present invention are administered as pharmaceuticals, to humans and animals, they can be given alone or as a pharmaceutical composition containing, for example, 0.001 to 90% (more preferably, 0.005 to 70%, such as 0.01 to 30%) of active ingredient in combination with a pharmaceutically acceptable carrier.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient that is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a composition of the invention will be that amount of the compound that is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a therapeutic composition may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Therapeutic compositions can be administered with medical devices known in the art. For example, in a preferred embodiment, a therapeutic composition of the invention can be administered with a needleless hypodermic injection device, such as the devices disclosed in

U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, the bispecific constructs of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. No. 4,522,811; 5,374,548; and 5,399,331. The liposomes may comprise one or more moieties that are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J.J. Killion; I.J. Fidler (1994) Immunomethods 4:273. In one embodiment of the invention, the therapeutic compounds of the invention are formulated in liposomes; in a more preferred embodiment, the liposomes include a targeting moiety. In a most preferred embodiment, the therapeutic compounds in the liposomes are delivered by bolus injection to a site proximal to the tumor or infection. The composition must be fluid to the extent that easy syringability exists. It must be stable under the conditions of manufacture and storage and must be preserved against the contaminating action of microorganisms such as bacteria and fungi.

The ability of a compound to inhibit cancer can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

The composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound may be orally administered, for example, with an inert diluent or an assimilable edible carrier.

### Nucleic Acids

Also provided herein are isolated nucleic acid molecules encoding the bispecific constructs, or binding domains thereof, as well as expression vectors comprising such nucleic acids and host cells comprising such expression vectors. In one embodiment, a nucleic acid molecule coding for any of the bispecific constructs described herein is provided. In another embodiment, the nucleic acid molecule is in the form of an expression vector. In another embodiment, the nucleic acid molecule is in the form of an expression vector which expresses the bispecific construct (or portion thereof) when administered to a subject in vivo.

In one embodiment, the nucleic acid molecule comprises a nucleotide sequence encoding the anti-ILT4 binding domain (or portion thereof), the anti-PD-1 binding domain (or portion thereof), or both binding domains of the bispecific constructs described herein. In another embodiment, the nucleic acid molecule comprises a nucleotide sequence encoding the heavy and light chain sequences of the bispecific construct, as set forth in SEQ ID NOs: 64 and 63, respectively, or an amino acid sequence at least 90% identical thereto (e.g., at least 90%, 91%, 92%, 93%, 94%, 95%, 96%, 97%, 98% or 99% identical to one or more of the aforementioned sequences). In another embodiment, the heavy and light chains are encoded by the nucleotide sequences as set forth in SEQ ID NOs: 66 and 65, respectively, or a nucleotide sequence at least 90% identical thereto (e.g., at least 90%, 91%, 92%, 93%, 94%, 95%, 96%, 97%, 98% or 99% identical to one or more of the aforementioned sequences).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

### Combination Therapies

The bispecific constructs described herein, can be administered in combination with an additional therapy, i.e., combined with other agents. The term “co-administered” as used herein includes any or all of simultaneous, separate, or sequential administration of the bispecific constructs described herein with adjuvants and other agents, including administration as part of a dosing regimen. For example, the combination therapy can include administering any of the bispecific constructs described herein with at least one or more additional therapeutic agents, such as anti-inflammatory agents, DMARDs (disease- modifying anti-rheumatic drugs), immunosuppressive agents, chemotherapeutics, radiation therapy, other antibodies, cytotoxins and/or drugs, as well as adjuvants, immunostimulatory agents and/or immunosuppressive agents.

Chemotherapeutic agents suitable for coadministration with the bispecific constructs, described herein in the treatment of tumors include, for example: taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Further agents include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine) and temozolomide.

Agents that delete or inhibit immunosuppressive activities, for example, by immune cells (for example regulatory T-cells, NKT cells, macrophages, myeloid-derived suppressor cells, immature or suppressive dendritic cells) or suppressive factors produced by the tumor or host cells in the local microenvironment of the tumor (for example, TGFβ, indoleamine 2,3 dioxygenase—IDO), may also be administered with the bispecific constructs described herein. Such agents include antibodies and small molecule drugs such as IDO inhibitors such as 1 methyl tryptophan or derivatives.

Suitable agents for coadministration with the bispecific constructs described herein for treatment of such immune disorders include for example, immunosuppressive agents such as rapamycin, cyclosporin and FK506; anti-TNF agents such as etanercept, adalimumab and infliximab; and steroids. Examples of specific natural and synthetic steroids include, for example: aldosterone, beclomethasone, betamethasone, budesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, paramethasone, prednisolone, prednisone, tixocortol and triamcinolone.

Suitable agents for coadministration with the bispecific constructs described herein for inducement or enhancement of an immune response include, for example, adjuvants and/or immunostimulatory agents, non-limiting examples of which have been disclosed hereinbefore. In one embodiment, the immunostimulatory agent is a TLR3 agonist, such as Poly IC.

As used herein, the term “immunostimulatory agent” includes, but is not limited to, compounds capable of stimulating antigen presenting cells (APCs), such as dendritic cells

(DCs) and macrophages. For example, suitable immunostimulatory agents for use in the present invention are capable of stimulating APCs, so that the maturation process of the APCs is accelerated, the proliferation of APCs is increased, and/or the recruitment or release of co-stimulatory molecules (e.g., CD80, CD86, ICAM-1, MHC molecules and CCR7) and pro-inflammatory cytokines (e.g., IL-1β, IL-6, IL-12, IL-15, and IFN-γ) is upregulated.

Suitable immunostimulatory agents are also capable of increasing T cell proliferation. Such immunostimulatory agents include, but are not be limited to, CD40 ligand; FLT 3 ligand; cytokines, such as IFN-α, IFN-β, IFN-γ and IL-2; colony-stimulating factors, such as G-CSF (granulocyte colony-stimulating factor) and GM-CSF (granulocyte-macrophage colony-stimulating factor); an agonistic CD40 antibody, CTLA-4 antibody, PD-1 antibody (i.e., a second anti-PD-1 antibody), 41BB antibody, or OX-40 antibody; LPS (endotoxin); ssRNA; dsRNA; Bacille Calmette-Guerin (BCG); Levamisole hydrochloride; and intravenous immune globulins.

In one embodiment an immunostimulatory agent may be a CD40 antibody having agonistic features. Such features include, for example, an increase in T-cell activity and/or an increase in B cell activation as measured, e.g., by an increase in the expression of cell surface markers selected from the group consisting of HLA-DR V450, CD54 PE, CD86 APC, CD83 BV510, CD19 V500, CD54 PE, HLA-DR V450, CD23 PerCP-Cy5.5, CD69 APC, CD86 APC, CD38 and CD71 PE. In another embodiment, the CD40 antibody blocks binding of CD40 to CD40L (CD154) on CD40-expressing cells and/or induces apoptosis of cells, as measured, e.g., by increased expression of CD95. Particular agonistic CD40 antibodies of the invention include antibodies described in WO2017/184619, such as the agonistic CD40 antibody 3C3 (CDX-1140).

In another embodiment an immunostimulatory agent may be a Toll-like Receptor (TLR) agonist. For example the immunostimulatory agent may be a TLR3 agonist such as double-stranded inosine: cytosine polynucleotide (Poly I: C, for example available as AmpligenTM from Hemispherx Bipharma, PA, US or Poly IC: LC from Oncovir) or Poly A: U; a TLR4 agonist such as monophosphoryl lipid A (MPL) or RC-529 (for example as available from GSK, UK); a TLR5 agonist such as flagellin; a TLR7 or TLR8 agonist such as an imidazoquinoline TLR7 or TLR 8 agonist, for example imiquimod (eg AldaraTM) or resiquimod and related imidazoquinoline agents (for example as available from 3M Corporation); or a TLR 9 agonist such as a deoxynucleotide with unmethylated CpG motifs (so-called “CpGs”, for example as available from Coley Pharmaceutical). Such immunostimulatory agents may be administered simultaneously, separately or sequentially with the bispecific constructs described herein.

### Uses and Methods of the Invention

Also provided herein are methods of methods of inducing or enhancing an immune response, and methods of treating cancer by administering the bispecific constructs or compositions described herein to a patient in need thereof.

The terms “inducing an immune response” and “enhancing an immune response” are used interchangeably and refer the stimulation of an immune response (i.e., either passive or adaptive) to a particular antigen.

The terms “treat,” “treating,” and “treatment,” as used herein, refer to therapeutic measures described herein. The methods of “treatment” employ administration to a subject, in need of such treatment, a bispecific construct or composition as described herein, for example, a subject in need of an enhanced immune response against a particular antigen or a subject who ultimately may acquire such a disorder, in order to cure, delay, reduce the severity of, or ameliorate one or more symptoms of the disorder or recurring disorder, or in order to prolong the survival of a subject beyond that expected in the absence of such treatment.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

The term “patient” includes human and other mammalian subjects that receive either prophylactic or therapeutic treatment.

As used herein, the term “inhibits growth” (e.g., referring to cells) is intended to include any measurable decrease in the growth of a cell, e.g., the inhibition of growth of a cell by at least about 10%, 20%, 30%, 40%, 50%, 60%, 70%, 80%, 90%, 99%, or 100%.

In another aspect, methods for inducing or enhancing an immune response (e.g., against an antigen) in a subject comprising administering to the subject any one of the bispecific constructs or the compositions described herein, in an amount effective to induce or enhance an immune response in the subject (e.g., against an antigen).

In another aspect, methods of for treating cancer in a subject are provided, the method comprising administering to the subject the bispecific constructs or the compositions described herein, in an amount effective to treat the condition or disease.

The subject can be, for example, one who suffers from a condition or disease in which stimulation of an immune response is desired. In one embodiment, the condition or disease is cancer. Types of cancers include, but are not limited to, leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin' s disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system. Particular cancers include ILT4-expressing tumors selected from the group consisting of chronic lymphocytic leukemia, mantle cell lymphoma, primary central nervous system lymphoma, Burkitt's lymphoma and marginal zone B cell lymphoma. Other disease indications include bacterial, fungal, viral and parasitic infectious diseases.

The methods of inducing or enhancing an immune response (e.g., against an antigen) in a subject described herein can further comprise administering the antigen to the subject. As used herein, the term “antigen” refers to any natural or synthetic immunogenic substance, such as a protein, peptide, hapten, polysaccharide and/or lipid. The bispecific construct or composition described herein and antigen can be administered at the same time or, alternatively, the bispecific construct or composition can be administered before or after the antigen is administered.

In one embodiment, a bispecific construct or composition described herein is administered in combination with a vaccine, to enhance the immune response against the vaccine antigen, for example a tumor antigen (to thereby enhance the immune response against the tumor) or an antigen from an infectious disease pathogen (to thereby enhance the immune response against the infectious disease pathogen). Accordingly, in one embodiment, a vaccine antigen can comprise, for example, an antigen or antigenic composition capable of eliciting an immune response against a tumor or against an infectious disease pathogen such as a virus, a bacteria, a parasite or a fungus. The antigen or antigens be derived from tumors, such as the various tumor antigens previously disclosed herein. Alternatively, the antigen or antigens can be derived from pathogens such as viruses, bacteria, parasites and/or fungi.

Preferred antigens to be co-administered with the bispecific constructs or the compositions described herein include tumor antigens and vaccine antigens (e.g., bacterial, viral or other pathogen antigens against which protective immunity is desired to be raised in a subject for purposes of vaccination). Additional examples of suitable pathogen antigens include tumor-associated antigens (TAAs), including but not limited to, sequences comprising all or part of the sequences of EGFR, EGFRVIII, gp100 or Pmel17, HER2/neu, mesothelin, CEA, MARTI, MAGE-A1, MAGE-A2, MAGE-A3, MAGE-A4, MUC-1, GPNMB, HMW-MAA, TIMI, RORI, CD19 and germ cell derived tumor antigens.

Other suitable antigens include viral antigens for the prevention or treatment of viral diseases. Examples of viral antigens include, but are not limited to, HIV-1 env, HBsAg, HPV, FAS, HSV-1, HSV-2, p17, ORF2 and ORF3 antigens. In addition, viral antigens or antigenic determinants can be derived from, for example,: Cytomegalovirus (especially Human, such as gB or derivatives thereof); Epstein Barr virus (such as gp350); flaviviruses (e.g. Yellow Fever Virus, Dengue Virus, Tick-borne encephalitis virus, Japanese Encephalitis Virus); hepatitis virus such as hepatitis B virus (for example Hepatitis B Surface antigen such as the PreSI, PreS2 and S antigens described in EP-A-414 374; EP-A-0304 578, and EP-A-198474), hepatitis A virus, hepatitis C virus and hepatitis E virus; HIV-1, (such as tat, nef, gp120 or gp160); human herpes viruses, such as gD or derivatives thereof or Immediate Early protein such as ICP27 from HSV1 or HSV2; human papilloma viruses (for example HPV6, 11, 16, 18); Influenza virus (whole live or inactivated virus, split influenza virus, grown in eggs or MDCK cells, or Vero cells or whole flu virosomes (as described by Gluck, Vaccine, 1992, 10, 915-920) or purified or recombinant proteins thereof, such as NP, NA, HA, or M proteins); measles virus; mumps virus; parainfluenza virus; rabies virus; Respiratory Syncytial virus (such as F and G proteins); rotavirus (including live attenuated viruses); smallpox virus; Varicella Zoster Virus (such as gpI, II and IE63); and the HPV viruses responsible for cervical cancer (for example the early proteins E6 or E7 in fusion with a protein D carrier to form Protein D-E6 or E7 fusions from HPV 16, or combinations thereof; or combinations of E6 or E7 with L2 (see for example WO 96/26277).

Examples of bacterial antigens include, but are not limited to, Toxoplasma gondii or Treponema pallidum. The bacterial antigens can be in the treatment or prevention of various bacterial diseases such as Anthrax, Botulism, Tetanus, Chlamydia, Cholera, Diphtheria, Lyme Disease, Syphilis and Tuberculosis. Bacterial antigens or antigenic determinants can be derived from, for example: Bacillus spp., including B. anthracis (e.g., botulinum toxin); Bordetella spp, including B. pertussis (for example pertactin, pertussis toxin, filamenteous hemagglutinin, adenylate cyclase, fimbriae); Borrelia spp., including B. burgdorferi (eg OspA, OspC, DbpA, DbpB), B. garinii (eg OspA, OspC, DbpA, DbpB), B. afzelii (eg OspA, OspC, DbpA, DbpB), B. andersonii (eg OspA, OspC, DbpA, DbpB), B. hermsii; Campylobacter spp, including C. jejuni (for example toxins, adhesins and invasins) and C. coli; Chlamydia spp., including C. trachomatis (eg MOMP, heparin-binding proteins), C. pneumonie (eg MOMP, heparin-binding proteins), C. psittaci; Clostridium spp., including C. tetani (such as tetanus toxin), C. botulinum (for example botulinum toxin), C. difficile (eg clostridium toxins A or B); Corynebacterium spp., including C. diphtheriae (eg diphtheria toxin); Ehrlichia spp., including E. equi and the agent of the Human Granulocytic Ehrlichiosis; Rickettsia spp, including R.rickettsii; Enterococcus spp., including E. faecalis, E. faecium; Escherichia spp, including enterotoxic E. coli (for example colonization factors, heat-labile toxin or derivatives thereof, or heat-stable toxin), enterohemorragic E. coli, enteropathogenic E. coli (for example shiga toxin-like toxin); Haemophilus spp., including H. influenzae type B (eg PRP), non-typable H. influenzae, for example OMP26, high molecular weight adhesins, P5, P6, protein D and lipoprotein D, and fimbrin and fimbrin derived peptides (see for example U.S. Pat. No. 5,843,464); Helicobacter spp, including H. pylori (for example urease, catalase, vacuolating toxin); Pseudomonas spp, including P. aeruginosa; Legionella spp, including L. pneumophila; Leptospira spp., including L. interrogans; Listeria spp., including L. monocytogenes; Moraxella spp, including M catarrhalis, also known as Branhamella catarrhalis (for example high and low molecular weight adhesins and invasins); Morexella Catarrhalis (including outer membrane vesicles thereof, and OMP106 (see for example W097/41731)); Mycobacterium spp., including M. tuberculosis (for example ESAT6, Antigen 85A,-B or -C), M. bovis, M. leprae, M. avium, M. paratuberculosis, M. smegmatis; Neisseria spp, including N. gonorrhea and N. meningitidis (for example capsular polysaccharides and conjugates thereof, transferrin-binding proteins, lactoferrin binding proteins, PilC, adhesins); Neisseria mengitidis B (including outer membrane vesicles thereof, and NspA (see for example WO 96/29412); Salmonella spp, including S. typhi, S. paratyphi, S. choleraesuis, S. enteritidis; Shigella spp, including S. sonnei, S. dysenteriae, S. flexnerii; Staphylococcus spp., including S. aureus, S. epidermidis; Streptococcus spp, including S. pneumonie (e.g., capsular polysaccharides and conjugates thereof, PsaA, PspA, streptolysin, choline-binding proteins) and the protein antigen Pneumolysin (Biochem Biophys Acta, 1989,67,1007; Rubins et al., Microbial Pathogenesis, 25,337-342), and mutant detoxified derivatives thereof (see for example WO 90/06951; WO 99/03884); Treponema spp., including T. pallidum (eg the outer membrane proteins), T. denticola, T. hyodysenteriae; Vibrio spp, including V. cholera (for example cholera toxin); and Yersinia spp, including Y. enterocolitica (for example a Yop protein), Y. pestis, Y. pseudotuberculosis.

Parasitic/fungal antigens or antigenic determinants can be derived from, for example,: Babesia spp., including B. microti; Candida spp., including C. albicans; Cryptococcus spp., including C. neoformans; Entamoeba spp., including E. histolytica; Giardia spp., including; G. lamblia; Leshmania spp., including L. major; Plasmodium. faciparum (MSP1, AMA1, MSP3, EBA, GLURP, RAP1, RAP2, Sequestrin, PfEMP1, Pf332, LSA1, LSA3, STARP, SALSA, PfEXPI, Pfs25, Pfs28, PFS27/25, Pfs16, Pfs48/45, Pfs230 and their analogues in Plasmodium spp.); Pneumocystis spp., including P. carinii; Schisostoma spp., including S. mansoni; Trichomonas spp., including T. vaginalis; Toxoplasma spp., including T. gondii (for example SAG2, SAG3, Tg34); Trypanosoma spp., including T. cruzi.

It will be appreciated that in accordance with this aspect of the present invention antigens and antigenic determinants can be used in many different forms. For example, antigens or antigenic determinants can be present as isolated proteins or peptides (for example in so-called “subunit vaccines”) or, for example, as cell-associated or virus-associated antigens or antigenic determinants (for example in either live or killed pathogen strains). Live pathogens will preferably be attenuated in known manner. Alternatively, antigens or antigenic determinants may be generated in situ in the subject by use of a polynucleotide coding for an antigen or antigenic determinant (as in so-called “DNA vaccination”), although it will be appreciated that the polynucleotides which can be used with this approach are not limited to DNA, and may also include RNA and modified polynucleotides as discussed above.

In one embodiment, a vaccine antigen can also be targeted, for example to particular cell types or to particular tissues. For example, the vaccine antigen can be targeted to Antigen Presenting Cells (APCs), for example by use of agents such as antibodies targeted to APC-surface receptors such as DEC-205, for example as discussed in WO 2009/061996 (Celldex Therapeutics, Inc), or the Mannose Receptor (CD206) for example as discussed in WO 03040169 (Medarex, Inc.).

### Kits

Also provided are kits (e.g., diagnostic kits) comprising one or more bispecific construct or composition as described herein, optionally with instructions for use. Kits may also include informative pamphlets, for example, pamphlets informing one how to use reagents to practice a method disclosed herein. The term “pamphlet” includes any writing, marketing materials or recorded material supplied on or with the kit, or which otherwise accompanies the kit.

The present invention is further illustrated by the following examples, which should not be construed as further limiting. The contents of figures and all references, patents and published patent applications cited throughout this application are expressly incorporated herein by reference.

### V. Examples

### Example 1: Generation of Bispecific Construct

Bispecific constructs were generated using antibodies (e.g., binding domains) targeting ILT4 and PD-1 to form the bispecific construct CDX-585. A tetravalent format (bivalent for each target) was developed using a fully human IgGI AQQYTE kappa backbone for the anti-PD1 antibody and a single chain Fv fragment (scFv) of the anti-ILT4 antibody genetically linked in VL-VH orientation to the C-terminus of the anti-PD1 antibody heavy chain. The Fe region of the heavy chain (HC) contained 6 additional amino acid modifications: L234A, L235Q, K322Q (AQQ) to eliminate all Fc interactions with Fe receptors, and M252Y, S254T, T256 (YTE) to extend serum half-life. To stabilize the scFv portion of CDX-585, a disulfide linkage was introduced between VL and VH by mutating the Gly to Cys at position 558 of the mature heavy chain (position VL100 according to the Kabat number system) and the Gly to Cys at position 629 of the mature heavy chain (position VH44 according to the Kabat number system).

DNA sequences encoding the light and heavy chains of CDX-585 were inserted into the pH2535-HDP glutamine synthetase (GS)-containing expression vector (Horizon Discovery) to construct a plasmid for the expression of CDX-585. The linearized plasmid was then transfected into the Horizon Discovery HD-BIOP3 cell line in which the endogenous glutamine synthetase (GS) gene has been knocked-out using recombinant Adeno-Associated Virus (rAAV) technology. Transfection pools were selected by growing in glutamine-free and animal component-free media and the bispecific construct product was purified from the pools by Protein A column chromatography.

The highest expressing pools were also single cell cloned (SCC) using the VIPS (Verified In-situ Plate Seeding) instrument. Research Cell Banks (RCB) were generated for the highest producing clones and again the resulting bispecific construct product was purified by Protein A column chromatography.

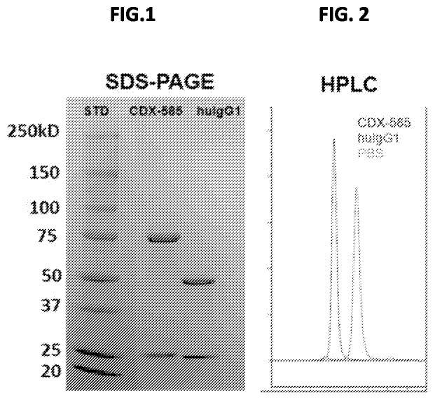

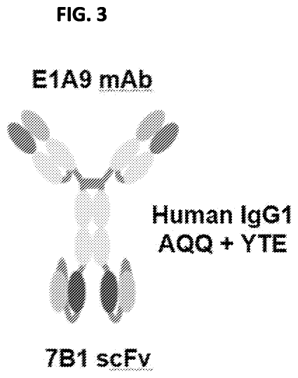

A schematic representation of the bispecific construct (CDX-585) is shown in FIG. 3. The purified construct was analysed by reducing SDS-PAGE on 4-15% Tris-HCL gel, and an image of the resulting gel is shown in FIG. 1 with an IgG1 comparator. The construct was also analysed by SEC-HPLC performed on TSK3000 column with 20 μg injections in PBS and the resulting trace is shown in FIG. 2 with an IgGI comparator.

### Example 2: Binding of CDX-585 to Human PD-1 using ELISA

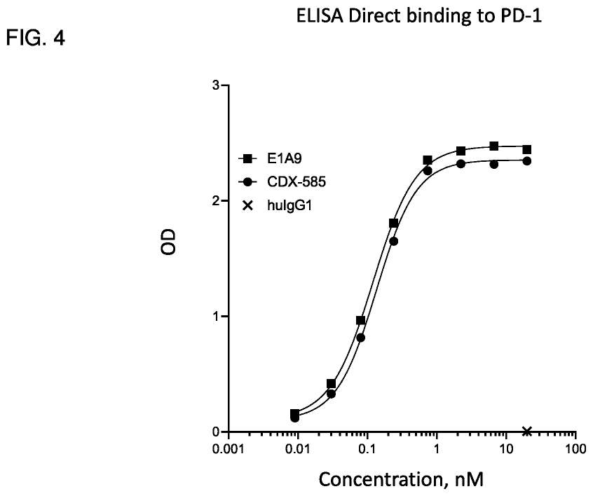

Microtiter plates were coated with recombinant human PD-1-msFc in PBS, and then blocked with 5% bovine serum albumin in PBS. Protein A purified anti-PD-1 monoclonal antibody E1A9, bispecific construct CDX-585 (from Example 1), and isotype control were added at various concentrations and incubated at 37° C. The plates were washed with PBS/Tween and then incubated with a goat-anti-human IgG Fc-specific polyclonal reagent conjugated to horseradish peroxidase at 37° C. After washing, the plates were developed with HRP substrate, and analyzed at OD 450 using a microtiter plate reader. A representative binding curve is shown in FIG. 4.

### Example 3: Binding of CDX-585 to Cells Expressing Human PD-1

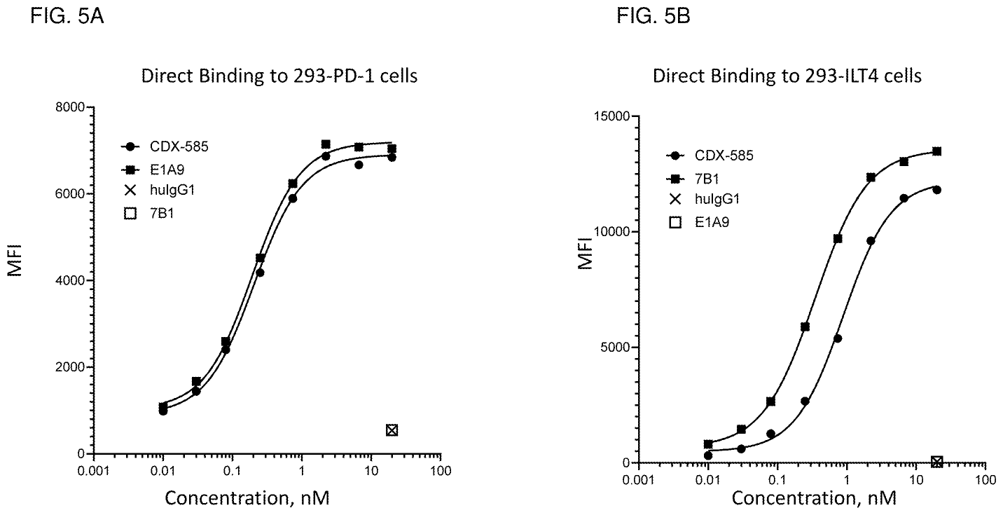

Protein A purified monoclonal antibodies 7B1 and E1A9, bispecific construct CDX-585 (from Example 1), and isotype control were incubated with HEK293 cells expressing human PD-1 at room temperature on a plate shaker. After 20 minutes, the cells were washed with PBS containing 0.1% BSA and 0.05% NaN₃ (PBA) and the bound antibodies were detected by incubating the cells with a PE labeled goat anti-human IgG Fc-specific probe. The excess probe was washed from the cells with PBA and the cell associated fluorescence was determined by analysis using a FACSCanto II™ instrument (BD Biosciences, NJ, USA) according to the manufacturer's directions. A representative binding curve is shown in FIG. 5A.

### Example 4: Binding of CDX-585 to Cells Expressing Human ILT4

Protein A purified monoclonal antibodies 7B1 and E1A9, bispecific construct CDX-585 (from Example 1), and isotype control were incubated with HEK293 cells expressing human ILT4 at room temperature on a plate shaker. After 20 minutes, the cells were washed with PBS containing 0.1% BSA and 0.05% NaN₃ (PBA) and the bound antibodies were detected by incubating the cells with a PE labeled goat anti-human IgG Fc-specific probe. The excess probe was washed from the cells with PBA and the cell associated fluorescence was determined by analysis using a FACSCanto II™ instrument (BD Biosciences, NJ, USA) according to the manufacturer's directions. A representative binding curve is shown in FIG. 5B.

### Example 5: Bifunctional Binding of CDX-585 to Cell-Expressed Human ILT4 and to Human PD-1

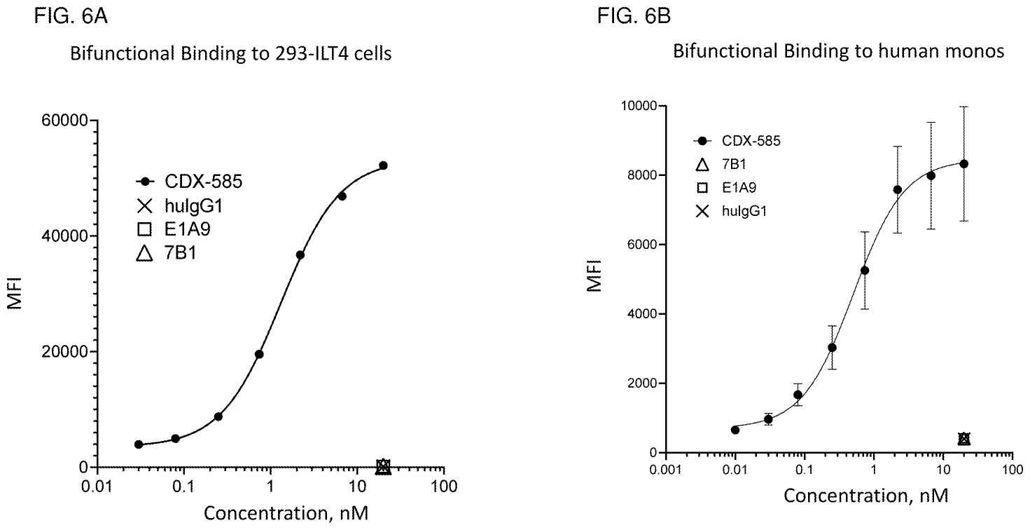

Binding of CDX-585 to ILT4 and PD-1 was assessed using HEK293 cells expressing human ILT4. In brief, dilutions of CDX-585 were allowed to bind to the ILT4 expressing cells before adding human PD-1-msFc that was detected with PE labeled goat anti-mouse IgG Fc-specific probe. A representative binding curve for CDX-585 is shown in FIG. 6A, which demonstrates significant binding to ILT4 and PD-1.

### Example 6: Bifunctional Binding of CDX-585 to Human Monocytes

Binding of CDX-585 to ILT4 and PD-1 was assessed using human monocytes. In brief, dilutions of CDX-585 were incubated with human PBMC's before staining for monocytes with an anti-CD14 APC labeled antibody. Human PD-1-msFc was added and detected with PE labeled goat anti-mouse IgG Fc-specific probe. A representative binding curve for CDX-585 is shown in FIG. 6B, which demonstrates significant binding to ILT4 and PD-1.

### Example 7: Determination of Affinity and Rate Constants of Human bsAbs by Bio-Layer Interferometry (BLI)

Binding affinity and binding kinetics of CDX-585 were analyzed by bio-layer interferometry (BLI) using an Octet™ QK^{e} instrument (Sartorius BioAnalytical Instruments) according to the manufacturer's guidelines. CDX-585 was captured on Anti-Human Fc Capture (AHC) biosensors (Sartorius). Each antibody was prepared in pH 7.2 kinetic assay dilution buffer and loaded on freshly hydrated and pre-conditioned AHC biosensors at 0.5 ug/mL for 300sec at 30° C. and 1000 rpm plate shake speed. For one assay, eight biosensors were loaded with the same antibody. Binding was determined by exposing seven of the antibody loaded biosensors to analyte: soluble human ILT4-HIS (Celldex in-house reagent) or human PD-1-HIS (R&D Systems). Affinity measurements were determined using 2-fold serial dilutions of analyte ranging from 50nM to 0.4nM in dilution buffer at 30° C. and 1000rpm plate shake speed. The appropriate dilution range for each antibody-antigen assay was determined experimentally. Association of the antibody loaded biosensors in analyte wells was carried out for 300 seconds, the biosensors were then moved to dilution buffer wells for 1500sec for dissociation measurements. Corresponding controls were conducted in each case by keeping the remaining biosensor with captured antibody in dilution buffer wells for association and dissociation steps. The data for the control biosensor were used to subtract background and account for biosensor drift and antibody dissociation from the biosensors. Octet BLI Analysis Software version 12.2.0.2 (Sartorius BioAnalytical Instruments) was used in each case to derive kinetic parameters from the concentration series of analyte in dilution buffer binding to captured antibody. The association and dissociation curves were fitted to a 1:1 binding model using the data analysis software according to the manufacturer's guidelines.

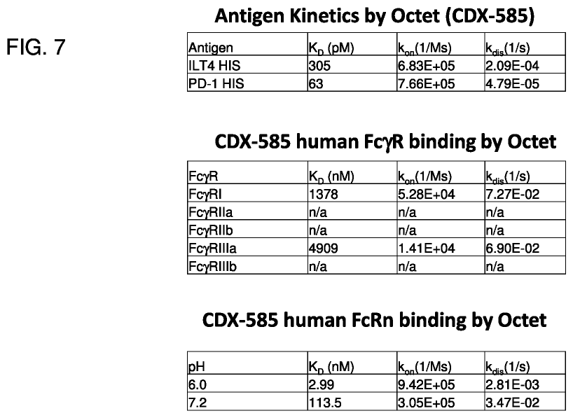

The affinity and kinetic parameters (with background subtracted) as determined are shown on FIG. 7, where kₒₙ=rate of association, k_{dis}=rate of dissociation, and K_{D}=affinity constant, determined by the ratio k_{dis}/kₒₙ.

### Example 8: Kinetic Analysis of CDX-585 binding to human Fcγ and FcRn Receptors

Affinity measurements were performed using an Octet™ QKe instrument (Sartorius BioAnalytical Instruments) according to the manufacturer's guidelines. Biotinylated human FcγRs and FcRn (Acro Biosystems) were captured on Streptavidin (SA) biosensors (Sartorius.) Each huFcγ receptor was prepared in pH 7.2 kinetic assay dilution buffer to 0.2 ug/ml and loaded on four SA biosensors for 300 seconds. Binding was determined by exposing two of the huFcγ receptor-loaded biosensors to CDX-585. Affinity measurements were determined using dilutions in pH 7.2 kinetic assay dilution buffer at 800 nM and 400 nM. The appropriate dilution range for each antibody-huFcγR assay was determined experimentally. For each huFcγR tested, a positive control well containing 100nM unmodified HuIgG1 was exposed to one huFcγR loaded sensor. Corresponding controls were conducted in each case with the remaining biosensor with captured huFcγ in dilution buffer for association and dissociation steps. Binding to huFcRn was determined for antibodies in kinetic assay dilution buffer at both pH 6.0 and pH 7.2. For one assay, eight biosensors were loaded with biotinylated huFcRn (Acro Biosystems) diluted to 0.3μg/ml in pH 7.2 kinetic assay dilution buffer. Binding was determined by exposing seven of the huFcRn loaded biosensors to CDX-585. Affinity measurements were determined using 2-fold serial dilutions of analyte ranging from 200 nM to 0.8 nM in the appropriate pH dilution buffer at 30° C. and 1000rpm plate shake speed. The appropriate dilution range for each buffer pH was determined experimentally. Association of the antibody loaded biosensors in analyte wells was carried out for 120 seconds, the biosensors were then moved to the corresponding pH dilution buffer wells for 180sec for dissociation measurements. Corresponding controls were conducted in each case by keeping the remaining biosensor with captured huFcRn in corresponding pH dilution buffer wells for association and dissociation steps. The data for the buffer control biosensor were used to subtract background and account for biosensor drift and antibody dissociation from the biosensors for both huFcγR and huFcRn assays. Octet BLI Analysis Software version 12.2.0.2 (Sartorius BioAnalytical Instruments) was used in each case to derive kinetic parameters from the concentration series of analyte in dilution buffer binding to captured huFcγR or FcRn. The association and dissociation curves were fitted to a 1:1 binding model using the data analysis software according to the manufacturer's guidelines.

The affinity and kinetic parameters (with background subtracted) as determined are shown on FIG. 7, where kₒₙ=rate of association, k_{dis}=rate of dissociation, and K_{D}=affinity constant, determined by the ratio k_{dis}/kₒₙ.

### Example 9: T cell PD1/PD-L1 Blockade Bioassay

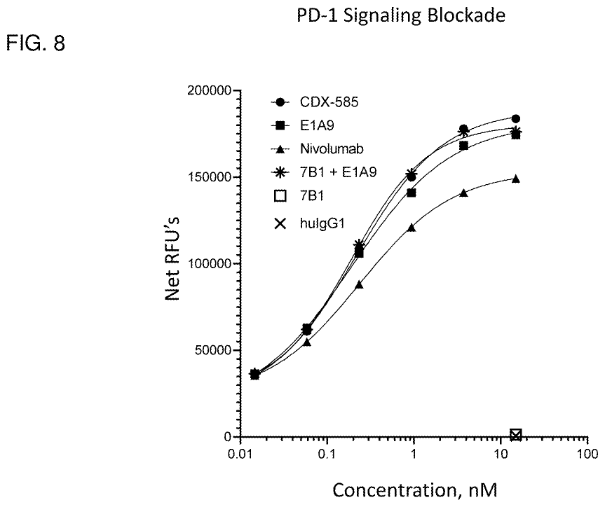

The effect of CDX-585 on blockade of the PD1/PD-L1 interaction was determined using the commercially available PD-1/PD-L1 Blockade Assay from Promega. Two engineered cell lines (PD1 Effector cells and PD-L1 aAPC/CHO-K1 cells) were co-cultured in the presence of the antibodies for 6 hours. Blocking of the PD1/PD-L1 interaction results in TCR activation and induces luminescence via the NFAT pathway. Luminescence was detected by the addition of Bio-Glo reagent and quantitated on a Perkin Elmer Victor X4 luminometer. As shown in FIG. 8, the anti-PD-1 antibody E1A9 and CDX-585 effectively block the PD1/PD-L1 interaction between cells leading to activation of the NFAT pathway.

### Example 10: Induction of TNF-α Production with CDX-585

Macrophages and dendritic cells were derived from human monocytes as follows: PBMC's were added to a T175cm² flasks and monocytes allowed to adhere for approximately 2 hours at 37° C., 6% CO₂. The non-adherent cells were removed and the monocytes cultured for 7 days in RPMI containing 10% FBS and 50 ng/ml M-CSF (R&D Systems) to prepare macrophages. Dendritic cells were prepared by culturing the monocytes for 7 days in RPMI containing 10% FBS, 100 ng/mL GM-CSF and 10 ng/ml IL-4 (R&D Systems)

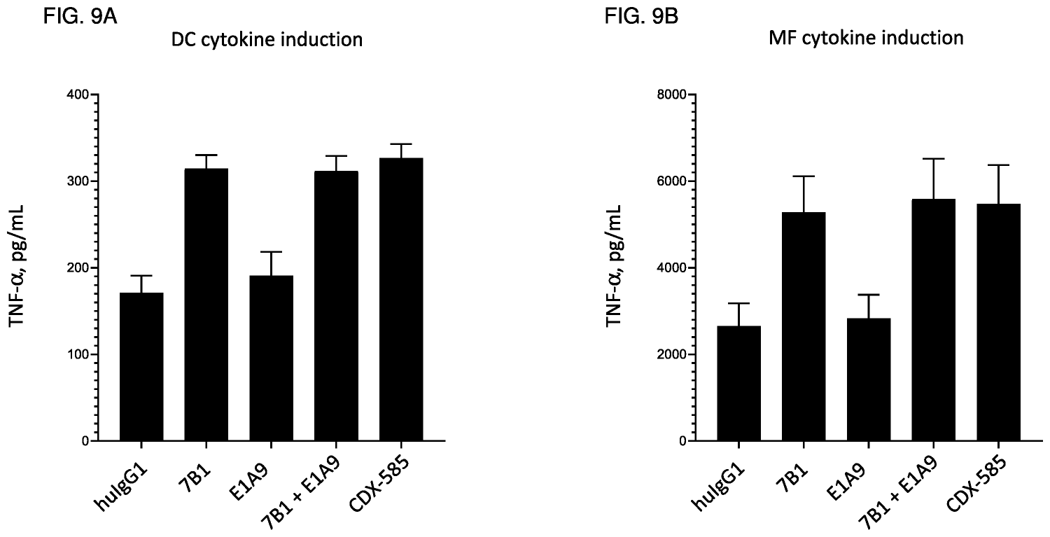

The cells were then incubated in the presence of CDX-585 and the appropriate antibody controls with 50 ng/mL LPS (Invivogen) at 37° C., 6% CO₂. After 24 hours, the cells were harvested and the supernatant was collected and stored for cytokine analysis. Induction of TNF-α was evaluated in the supernatants collected by ELISA (R&D Systems). The increase in TNF-α production is shown in FIGS. 9A and 9B.

### Example 11: Inhibition of HLA-G Binding to ILT4 by CDX-585

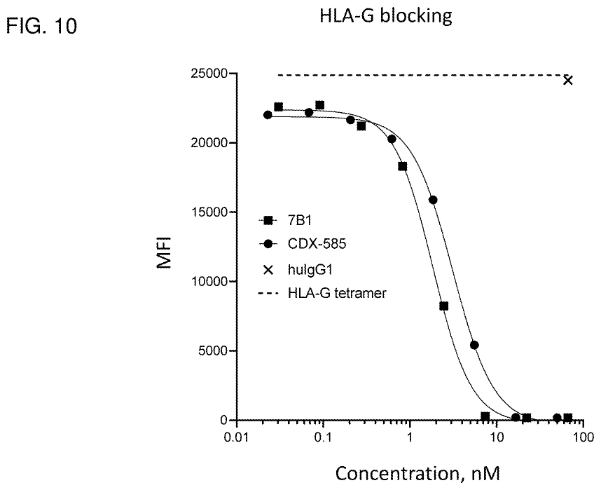

Dilutions of CDX-585 and antibody controls were incubated on HEK293 cells expressing human ILT4 at room temperature on a plate shaker. After 30 minutes, the cells were washed and PE-labeled HLA-G tetramer (FredHutch) was added. After an additional 30 minutes, the cells were washed with PBA and the cell associated fluorescence was determined by analysis using a FACSCanto II™ instrument (BD Biosciences, NJ, USA) according to the manufacturer's directions. Representative blocking curves are shown in FIG. 10.

### Example 12: M1 Macrophage Polarization with CDX-585

Macrophages were prepared as described in Example 10. Macrophages were cultured for 6 days in the presence of M-CSF along with 6.7 nM CDX-585 and antibody controls. LPS at 50 ng/ml (Invitrogen) was added overnight and the cells were harvested for analysis. Supernatant was also harvested for cytokine analysis.

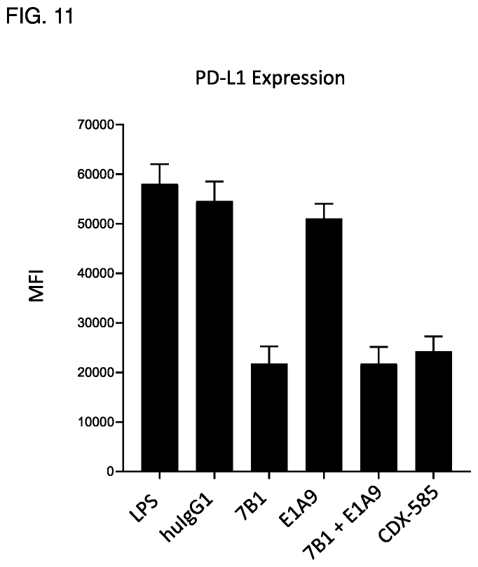

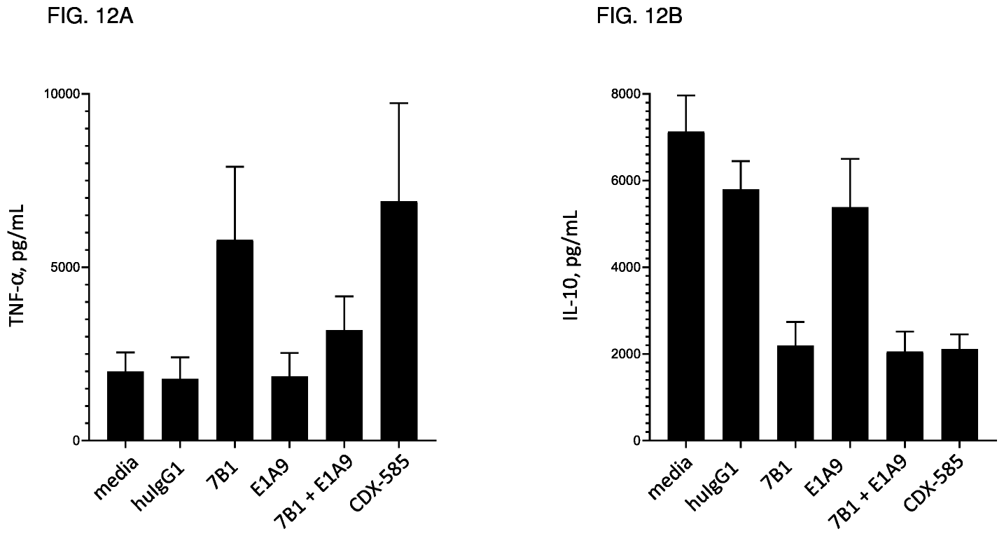

Cells were stained with a PE labeled anti-PD-L1 antibody then washed with PBA and the cell associated fluorescence was determined by analysis using a FACSCanto II™ instrument (BD Biosciences, NJ, USA) according to the manufacturer's directions. The downregulation of PD-L1 expression is shown on FIG. 11. In addition, supernatants collected were evaluated for TNF-α and IL-10 induction by ELISA (R&D Systems). FIG. 12A shows the increase in TNF-a production with CDX-585 and FIG. 12B demonstrates the downregulation IL-10 secretion.

### Example 13: T cell Activation via Mixed Lymphocyte Response

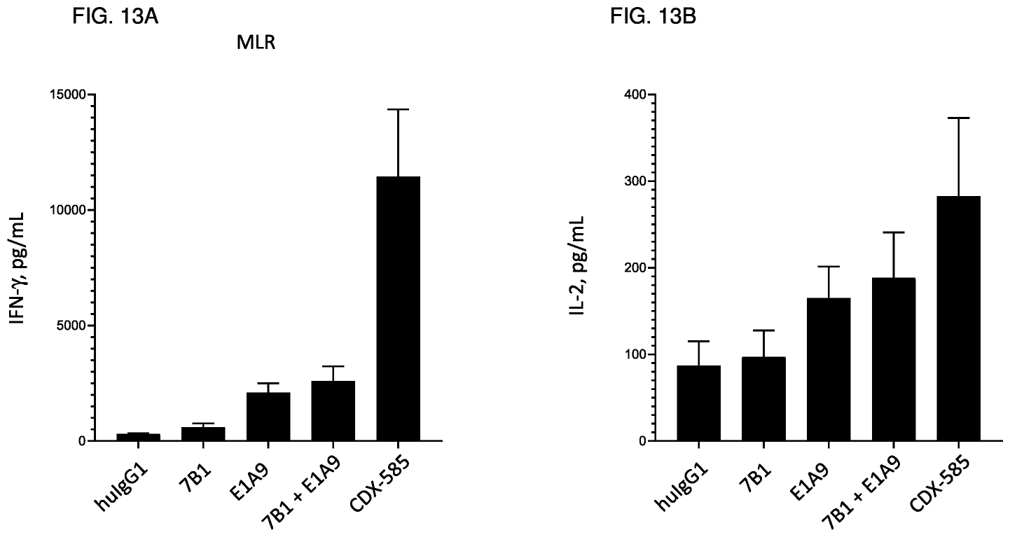

Human peripheral blood mononuclear cells were isolated from buffy coats using Ficoll separation, and CD4⁺ cells were further isolated from the PBMC's using magnetic bead separation technology from Miltenyi Biotec. Allogeneic dendritic cells were generated as follows: PMBC's were added to a T175cm² flasks and monocytes allowed to adhere for approximately 2 hours at 37° C., 6% CO₂. The non-adherent cells were removed and the monocytes cultured for 7 days in RPMI containing 10% FBS, 10 ng/ML IL-4 (R&D Systems) and 100 ng/mL GM-CSF (R&D Systems). The CD4⁺ cells and DC's were co-incubated and a 10:1 ratio in the presence of the antibody dilutions for 4 days. Supernatents were harvested and analyzed for IFN-γ and IL-2 production by ELISA (R&D Systems). As shown in FIGS. 13A and 13B, CDX-585 was able to induce a significant mixed lymphocyte response.

### Example 14: T cell Activation by CDX-585 with Dual Inhibition of ILT4 and PD-1

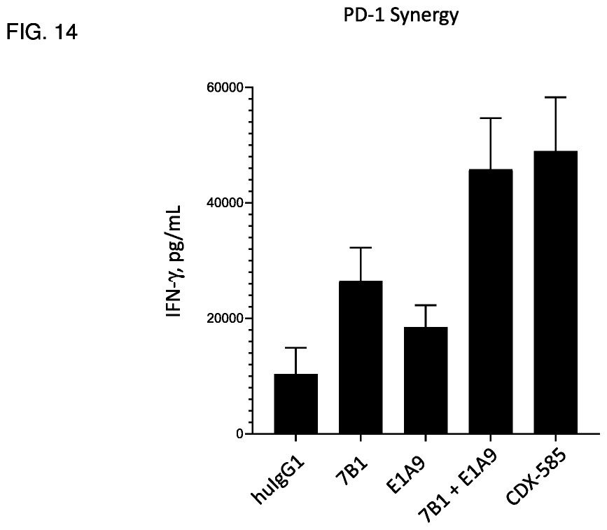

Human PBMC's were incubated overnight with 33 nM CDX-585 and antibody controls. A sub-optimal dose of anti-CD3 antibody (OKT3, eBioscience) was added and the cells incubated for an additional 3 days. Supernatant was harvested and IFN-g induction was evaluated. As shown in FIG. 14, both the CDX-585 bispecific and the combination of the individual antibodies increased IFN-g, indicating a synergistic effect of the combination of

ILT4 and PD-1 antibodies.

### Example 15: In vivo Antitumor Activity in Mouse Tumor Model

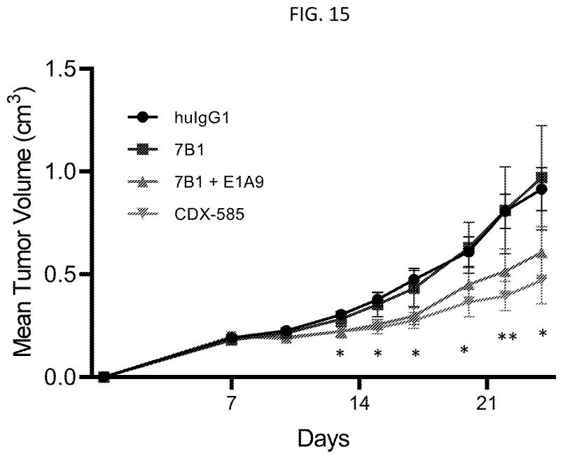

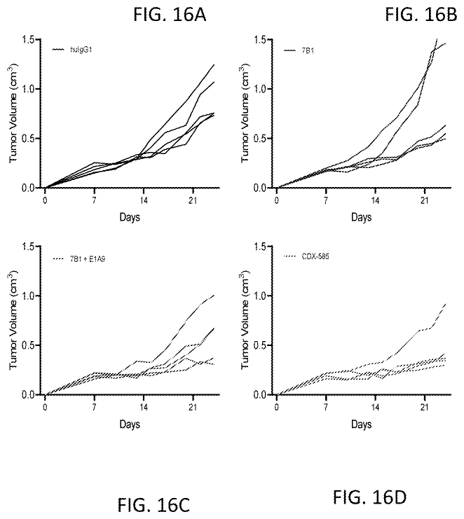

Twenty HuCD34-NCG mice (Charles River Laboratories), from two donors, were divided into four groups of five mice each. Mice were implanted with 2×10⁷ SKMEL-5 cells subcutaneously. Starting on the day following implantation, mice were treated as follows: Group 1: Human IgGI AQQ (0.5 mg/mouse), Group 2: CDX-585 (0.5 mg/mouse), Group 3: 7B 1 (0.375 mg/mouse), and Group 4: E1A9 (0.375 mg/mouse) and 7B1 (0.375mg/mouse). Mice were dosed once a week for 5 weeks. Tumor volumes were measured periodically. Statistical significance vs hulgG1 control was measured by student's T-test. Results are shown in FIGS. 15 and 16, where *=p<0.05, **=p<0.01.

### Example 16: Pilot Study in Cynomolgus Macaques

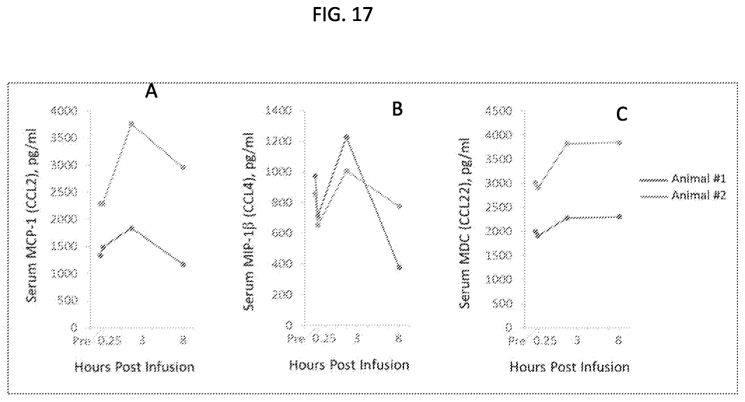

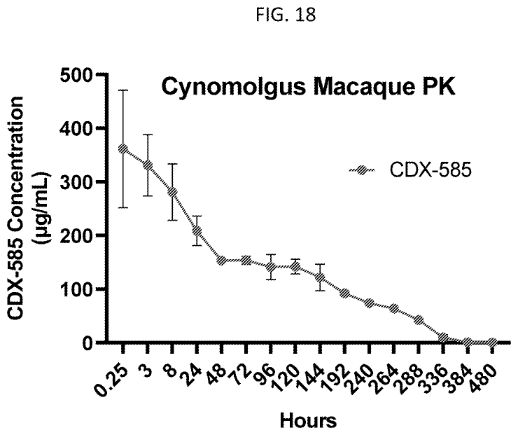

Cynomolgus macaques were given single a i.v. dose (10 mg/kg) of CDX-585. Serum concentrations of CDX-585 were measured by ELISA, and serum concentrations of cytokine/chemokines were analyzed by MesoScale Discovery (MSD). There were no significant adverse laboratory or clinical findings. Results are shown in FIGS. 17 and 18.

### Example 17: T Cell Activation via Mixed Lymphocyte Response

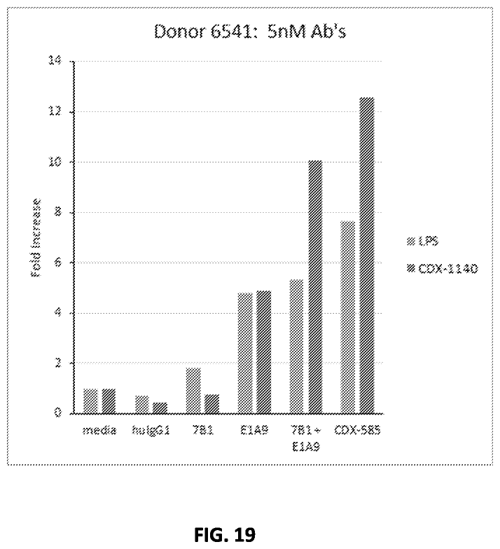

Human peripheral blood mononuclear cells were isolated from buffy coats using Ficoll separation, and CD4⁺ cells were further isolated from the PBMC's using magnetic bead separation technology from Miltenyi Biotec. Allogeneic dendritic cells (DCs) were generated as follows: PMBC's were added to a T175cm² flasks and monocytes allowed to adhere for approximately 2 hours at 37° C., 6% CO₂. The non-adherent cells were removed and the monocytes cultured for 7 days in RPMI containing 10% FBS, 10 ng/ml IL-4 (R&D Systems) and 100 ng/mL GM-CSF (R&D Systems). The DCs were incubated overnight with either 50 ng/mL LPS or 0.5 mg/mL of anti-CD40 antibody (CDX-1140) described in WO2017/184619. The pre-treated DCs were washed and co-incubated with the CD4⁺ cells at a 10:1 ratio in the presence of the antibodies at 5 mg/mL for 4 days. Supernatants were harvested and analyzed for IFN-g production by ELISA (R&D Systems). Results are shown in FIG. 19.

### Example 18: In vivo Antitumor Activity in mouse tumor model

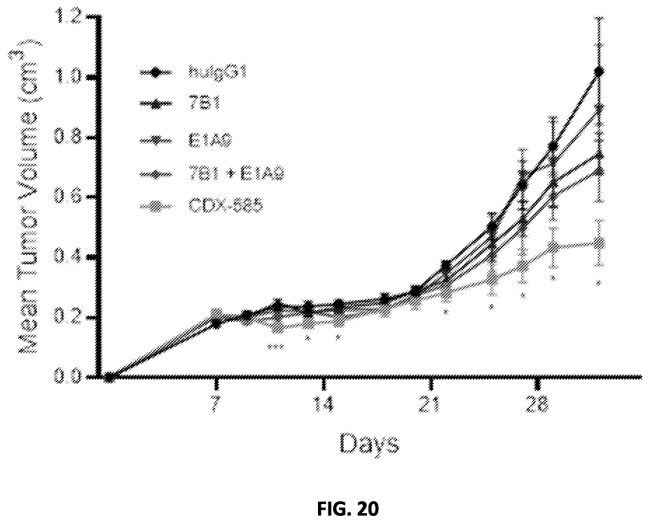

The mouse tumor model of Example 15 was repeated with additional controls. Results are shown in FIG. 20.

### SUMMARY OF SEQUENCE LISTING

TABLE 1
7A3 and 7B1 Humanized Sequences

| | |
|---|---|
| SEQ ID NO: 1 | GYTIH |
| 7A3 V_{H} CDR1 | |
| (Kabat) | |
| SEQ ID NO: 2 | GYSFTGY |
| 7A3 V_{H} CDR1 | |
| (Chothia) | |
| SEQ ID NO: 3 | LINPYTGGTDYNQKFKG |
| 7A3 V_{H} CDR2 | |
| (Kabat) | |
| SEQ ID NO: 4 | NPYTGG |
| 7A3 V_{H} CDR2 | |
| (Chothia) | |
| SEQ ID NO: 5 | ERPGGSQFIYYYPMDY |
| 7A3 V_{H} CDR3 | |
| SEQ ID NO: 6 | RASANIYSYLA |
| 7A3 V_{L} CDR1 | |
| SEQ ID NO: 7 | NAITLAE |
| 7A3 VL CDR2 | |
| SEQ ID NO: 8 | QHHYGTPFT |
| 7A3 VL CDR3 | |
| SEQ ID NO: 9 | QVQLVQSGAEVKKPGASVKVSCKASGYSFTGYTMHWVRQAPGQGLE |
| 7A3 V_{H} | WMGLINPYTGGTDYNQKFQGRVTMTVDKSTSTAYMELSSLRSEDTAV |
| Hu-VH2 | YYCARERPGGSQFIYYYPMDYWGQGTTVTVSS |
| SEQ ID NO: 10 | DIQMTQSPSSLSASVGDRVTITCRASANIYSYLAWYQQKPGKAPKFLVY |
| 7A3 VL | NAITLAEGVPSRFSGSGSGTDFTLTISSLQPEDFATYYCQHHYGTPFTFG |
| Hu-VL1 | GGTKLEIK |
| SEQ ID NO: 11 | GYTMH |
| 7B1 V_{H} CDR1 | |
| (Kabat) | |
| SEQ ID NO: 12 | GYSFTGY |
| 7B1 V_{H} CDR1 | |
| (Chothia) | |
| SEQ ID NO: 13 | LINPYTGGTDYNQKFKG |
| 7B1 V_{H} CDR2 | |
| (Kabat) | |
| SEQ ID NO: 14 | NPYTGG |
| 7B1 V_{H} CDR2 | |
| (Chothia) | |
| SEQ ID NO: 15 | ERPGGSQFIYYYALDY |
| 7B1 V_{H} CDR3 | |
| SEQ ID NO: 16 | RASENIYSYLA |
| 7B1 VL CDR1 | |
| SEQ ID NO: 17 | NADTLAE |
| 7B1 VL CDR2 | |
| SEQ ID NO: 18 | QHHYGTPFT |
| 7B1 VL CDR3 | |
| SEQ ID NO: 19 | QVQLVQSGAEVKKPGASVKVSCKASGYSFTGYTMHWVRQAPGQGLE |
| 7B1 V_{H} | WMGLINPYTGGTDYNQKFQGRVTMTVDRSTSTAYMELSSLRSEDTAV |
| Hu-VH2 | YYCARERPGGSQFIYYYALDYWGQGTTVTVSS |
| SEQ ID NO: 20 | DIQMTQSPSSLSASVGDRVTITCRASENIYSYLAWYQQKPGKAPKFLVY |
| 7B1 VL | NADTLAEGVPSRFSGSGSGTDFTLTISSLQPEDFATYYCQHHYGTPFTFG |
| Hu-VL1 | GGTKLEIK |
| SEQ ID NO: 21 | GY T (I,M) H |
| VH CDR1 | |
| Consensus | |
| SEQ ID NO: 22 | ERPGGSQFIY Y Y (P,A) (M,L) DY |
| VH CDR3 | |
| Consensus | |
| SEQ ID NO: 23 | RAS (A,E) NIYSYLA |
| VL CDR1 | |
| Consensus | |
| SEQ ID NO: 24 | NA (I,D) TLAE |
| VL CDR2 | |
| Consensus | |
| SEQ ID NO: 25 | QVQLVQSGAEVKKPGASVKVSCKASGYSFTGYTMHWVRQAPGQGLEWMG |
| 7A3 AQQ HC | LINPYTGGTDYNQKFQGRVTMTVDKSTSTAYMELSSLRSEDTAVYYCARER |
| amino acids | PGGSQFIYYYPMDYWGQGTTVTVSSASTKGPSVFPLAPSSKSTSGGTAALGCLV |
| | KDYFPEPVTVSWNSGALTSGVHTFPAVLQSSGLYSLSSVVTVPSSSLGTQTYICNVN |
| | 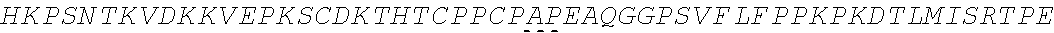 |
| | VTCVVVDVSHEDPEVKFNWYVDGVEVHNAKTKPREEQYNSTYRVVSVLTVLHQD |
| | 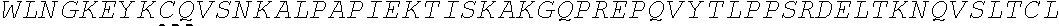 |
| | VKGFYPSDIAVEWESNGQPENNYKTTPPVLDSDGSFFLYSKLTVDKSRWQQGNVF |
| | SCSVMHEALHNHYTQKSLSLSPG |
| SEQ ID NO: 26 | DIQMTQSPSSLSASVGDRVTITCRASANIYSYLAWYQQKPGKAPKFLVYNAI |
| 7A3 LC amino | TLAEGVPSRFSGSGSGTDFTLTISSLQPEDFATYYCQHHYGTPFTFGGGTKL |
| acids | EIKRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| SEQ ID NO: 27 | QVQLVQSGAEVKKPGASVKVSCKASGYSFTGYTMHWVRQAPGQGLEWMG |
| 7B1 AQQ HC | LINPYTGGTDYNQKFQGRVTMTVDRSTSTAYMELSSLRSEDTAVYYCARER |
| amino acids | PGGSQFIYYYALDYWGQGTTVTVSSASTKGPSVFPLAPSSKSTSGGTAALGCLV |
| | KDYFPEPVTVSWNSGALTSGVHTFPAVLQSSGLYSLSSVVTVPSSSLGTQTYICNVN |
| | 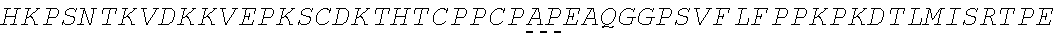 |
| | VTCVVVDVSHEDPEVKFNWYVDGVEVHNAKTKPREEQYNSTYRVVSVLTVLHQD |
| | 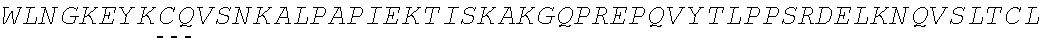 |
| | VKGFYPSDIAVEWESNGQPENNYKTTPPVLDSDGSFFLYSKLTVDKSRWQQGNVF |
| | SCSVMHEALHNHYTQKSLSLSPG |
| SEQ ID NO: 28 | DIQMTQSPSSLSASVGDRVTITCRASENIYSYLAWYQQKPGKAPKFLVYNA |
| | DTLAEGVPSRFSGSGSGTDFTLTISSLQPEDFATYYCQHHYGTPFTFGGGTK |
| 7B1 LC amino | LEIKRTVAAPSVFIFPPSDEQLKSGTASVVCLLNNFYPREAKVQWKVDNALQSGNS |
| acids | QESVTEQDSKDSTYSLSSTLTLSKADYEKHKVYACEVTHQGLSSPVTKSFNRGEC |

### PD-1 Antibody Sequences

TABLE 2A
VH CDRs-PD1-HuAb-E1A9C8A7-V8-3

| Region | Definition | Amino Acid Sequence |
|---|---|---|
| CDR-H1 | | |
| SEQ ID NO: 29 | Chotbia | GYTFTDY--- |
| SEQ ID NO: 30 | AbM | GYTFTDYVMH |
| SEQ ID NO: 31 | Kabat | -----DYVMH |
| SEQ ID NO: 32 | Contact | ----TDYVMH |
| SEQ ID NO: 33 | IMGT | GYTFTDYV-- |
| CDR-H2 | | |
| SEQ ID NO: 34 | Chothia | -----STYHIP--------- |
| SEQ ID NO: 35 | AbM | ---VISTYHIPAV------- |
| SEQ ID NO: 36 | Kabat | ---VISTYHIPAVYNQKFKG |
| SEQ ID NO: 37 | Contact | WMGVISTYHIPAV------- |
| SEQ ID NO: 38 | IMGT | ----ISTYHIPA-------- |
| CDR-H3 | | |
| SEQ ID NO: 39 | Chothia | --EVYGESYYFDV |
| SEQ ID NO: 40 | AbM | --EVYGESYYFDV |
| SEQ ID NO: 41 | Kabat | --EVYGESYYFDV |
| SEQ ID NO: 42 | Contact | AREVYGESYYFD- |
| SEQ ID NO: 43 | IMGT | AREVYGESYYFDV |

TABLE 2B
VL CDRs-PD1-HuAb-E1A9C8A7-V8-3

| Region | Definition | Amino Acid Sequence |
|---|---|---|
| CDR-L1 | | |
| SEQ ID NO: 44 | Chothia | RSSQSIVHSAGNTYLE-- |
| SEQ ID NO: 45 | AbM | RSSQSIVHSAGNTYLE-- |
| SEQ ID NO: 46 | Kabat | RSSQSIVHSAGNTYLE-- |
| SEQ ID NO: 47 | Contact | ------VHSAGNTYLEWY |
| SEO ID NO: 48 | IMGT | ----SIVHSAGNTY---- |
| CDR-L2 | | |
| SEQ ID NO: 49 | Chothia | ----KVSNRFS |
| SEQ ID NO: 50 | AbM | ----KVSNRFS |
| SEQ ID NO: 51 | Kabat | ----KVSNRFS |
| SEQ ID NO: 52 | Contact | LLIYKVSNRF- |
| SEQ ID NO: 53 | IMGT | ----KV----- |
| CDR-L3 | | |
| SEQ ID NO: 54 | Chothia | FQGSHVPYT |
| SEQ ID NO: 55 | AbM | FQGSHVPYT |
| SEQ ID NO: 56 | Kabat | FQGSHVPYT |
| SEQ ID NO: 57 | Contact | FQGSHVPY- |
| SEQ ID NO: 58 | IMGT | FQGSHVPYT |

TABLE 2C
VH CDRs-PD1-HuAb-E1A9C8A7-V8

| Region | Definition | Amino Acid Sequence |
|---|---|---|
| CDR-H1 | | |
| SEQ ID NO: 67 | Chothia | GYTFTDY--- |
| SEQ ID NO: 68 | AbM | GYTFTDYVMH |
| SEQ ID NO: 69 | Kabat | -----DYVMH |
| SEQ ID NO: 70 | Contact | ----TDYVMH |
| SEQ ID NO: 71 | IMGT | GYTFTDYV-- |
| CDR-H2 | | |
| SEQ ID NO: 72 | Chothia | -----STYHIP--------- |
| SEQ ID NO: 73 | AbM | ---VISTYHIPAV------- |
| SEQ ID NO: 74 | Kabat | ---VISTYHIPAVYNQKFKG |
| SEQ ID NO: 75 | Contact | WMGVISTYHIPAV------- |
| SEQ ID NO: 76 | IMGT | ----ISTYHIPA-------- |
| CDR-H3 | | |
| SEQ ID NO: 77 | Chothia | --EVYGDSYYFDV |
| SEQ ID NO: 78 | AbM | --EVYGDSYYFDV |
| SEQ ID NO: 79 | Kabat | --EVYGDSYYFDV |
| SEQ ID NO: 80 | Contact | AREVYGDSYYFD- |
| SEQ ID NO: 81 | IMGT | AREVYGDSYYFDV |

TABLE 2D
VL CDRs-PD1-HuAb-E1A9C8A7-V8

| Region | Definition | Amino Acid Sequence |
|---|---|---|
| CDR-L1 | | |
| SEQ ID NO: 82 | Chothia | RSSQSIVHSNGNTYLE-- |
| SEQ ID NO: 83 | AbM | RSSQSIVHSNGNTYLE-- |
| SEQ ID NO: 84 | Kabat | RSSQSIVHSNGNTYLE-- |
| SEQ ID NO: 85 | Contact | ------VHSNGNTYLEWY |
| SEQ ID NO: 86 | IMGT | ---QSIVHSNGNTY---- |
| CDR-L2 | | |
| SEQ ID NO: 87 | Chothia | ----KVSNRFS |
| SEQ ID NO: 88 | AbM | ----KVSNRFS |
| SEQ ID NO: 89 | Kabat | ----KVSNRFS |
| SEQ ID NO: 90 | Contact | LLIYKVSNRF- |
| SEQ ID NO: 91 | IMGT | ----KV----- |
| CDR-L3 | | |
| SEQ ID NO: 92 | Chothia | FQGSHVPYT |
| SEQ ID NO: 93 | AbM | FQGSHVPYT |
| SEQ ID NO: 94 | Kabat | FQGSHVPYT |
| SEQ ID NO: 95 | Contact | FQGSHVPY- |
| SEQ ID NO: 96 | IMGT | FQGSHVPYT |

TABLE 2E
VH/VL Sequences

| | |
|---|---|
| VH | Amino Acid Sequence (PD1-HuAb-E1A9C8A7-V8-3) |
| SEQ ID NO: 59 | QVQLVQSGAEVVKPGASVKISCKASGYTFTDYVMHWVRQAPGQSLEWM |
| | GVISTYHIPAVYNQKFKGKATMTVDTSTSTVYLELSSLRSEDTAVYYCAR |
| | EVYGESYYFDVWGQGTTVTVSS |
| VL | Amino Acid Sequence (PD1-HuAb-E1A9C8A7-V8-3) |
| SEQ ID NO: 60 | DIVMTQTPLSLPVTPGEPASISCRSSQSIVHSAGNTYLEWYLQKPGQSPQL |
| | LIYKVSNRFSGVPDRFSGSGSGTDFTLKISRVEAEDVGVYYCFQGSHVPYT |
| | FGGGTKVEIK |
| VH | Amino Acid Sequence (PD1-HuAb-E1A9C8A7-V8) |
| SEQ ID NO: 61 | QVQLVQSGAEVVKPGASVKISCKASGYTFTDYVMHWVRQAPGQSLEWM |
| | GVISTYHIPAVYNQKFKGKATMTVDTSTSTVYLELSSLRSEDTAVYYCAR |
| | EVYGDSYYFDVWGQGTTVTVSS |
| VL | Amino Acid Sequence (PD1-HuAb-E1A9C8A7-V8) |
| SEQ ID NO: 62 | DIVMTQTPLSLPVTPGEPASISCRSSQSIVHSNGNTYLEWYLQKPGQSPQL |
| | LIYKVSNRFSGVPDRFSGSGSGTDFTLKISRVEAEDVGVYYCFQGSHVPYT |
| | FGGGTKVEIK |

TABLE 3
Bispecific Construct Sequences

| Region | Amino Acid Sequence |
|---|---|
| SEQ ID NO: 63 | Variable region (underlined) |
| CDX-585 | Constant domains (double underlined) |
| LC amino acids | |
| | DIVMTQTPLSLPVTPGEPASISCRSSQSIVHSAGNTYLEWYLQKPGQSPQ |
| | LLIYKVSNRFSGVPDRESGSGSGTDFTLKISRVEAEDVGVYYCFQGSHV |
| | PYTFGGGTKVEIKRTVAAPSVFIFPPSDEQLKSGTASVVCLLNNFYPREA |
| | KVQWKVDNALQSGNSQESVTEQDSKDSTYSLSSTLTLSKADYEKHKV |
| | YACEVTHQGLSSPVTKSFNRGEC* |
| SEQ ID NO: 64 | Variable region (underlined) |
| CDX-585 | Constant domains (double underlined) |
| HC amino acids | scFv (broken underlined) |
| | underlined: Linkers (bold and italicized) |
| | modified bases (bolded) |
| | QVQLVQSGAEVVKPGASVKISCKASGYTFTDYVMHWVRQAPGQSLE |
| | WMGVISTYHIPAVYNQKFKGKATMTVDTSTSTVYLELSSLRSEDTAVY |
| | YCAREVYGESYYFDVWGQGTTVTVSSASTKGPSVFPLAPSSKSTSGGT |
| | AALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEAQG |
| | GPSVFLFPPKPKDTLYITREPEVTCVVVDVSHEDPEVKENWYVDGVEV |
| | HNAKTKPREEQYNSTYRVVSVLTVLHQDWLNGKEYKCQVSNKALPAP |
| | IEKTISKAKGQPREPQVYTLPPSRDELTKNQVSLTCLVKGFYPSDIAVEW |
| | ESNGQPENNYKTTPPVLDSDGSFFLYSKLTVDKSRWQQGNVESCSVMH |
| | 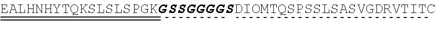 |
| | 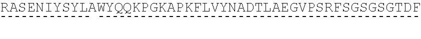 |
| | 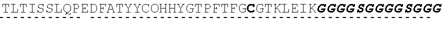 |
| | 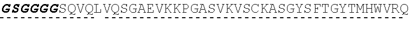 |
| | 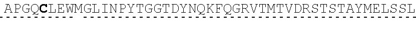 |
| | 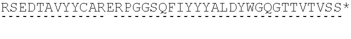 |
| Region | Nucleotide Sequence |
| SEQ ID NO: 65 | Variable region (underlined) |
| E1A9-7B1 | |
| (CDX-585) | Constant domains (double underlined) |
| LC nucleic acids | GACATCGTGATGACCCAGACACCATTGTCGTTGCCTGTTACTCCAGGAGAACC |
| | CGCATCAATTAGCTGCAGAAGCAGCCAGTCCATTGTGCATAGTGCCGGTAACA |
| | CCTATCTGGAGTGGTACCTACAGAAGCCCGGCCAGTCTCCTCAGCTTCTTATCT |
| | ACAAAGTGAGCAATAGGTTTTCTGGGGTGCCTGATCGGTTTAGCGGTAGCGGC |
| | TCCGGCACCGACTTTACACTGAAGATCAGCAGGGTGGAGGCTGAGGACGTTGG |
| | AGTGTACTACTGTTTTCAGGGCAGCCACGTGCCTTATACATTTGGTGGCGGCA |
| | CAAAGGTCGAGATCAAACGTACGGTGGCCGCTCCAAGCGTGTTCATCTTTCCC |
| | CCTTCTGACGAGCAGCTGAAGTCTGGCACAGCCTCCGTGGTGTGCCTGCTGAA |
| | CAACTTCTACCCCAGAGAGGCCAAGGTGCAGTGGAAGGTGGATAACGCTCTGC |
| | AGTCTGGCAATTCCCAGGAGAGCGTGACCGAGCAGGACTCTAAGGATTCCACA |
| | TATAGCCTGAGCTCTACCCTGACACTGTCTAAGGCCGATTACGAGAAGCACAA |
| | GGTGTATGCTTGCGAGGTGACCCATCAGGGCCTGTCCAGCCCAGTGACAAAGT |
| | CCTTCAATCGCGGCGAGTGTtga |
| SEQ ID NO: 66 | Variable region (underlined) |
| E1A9-7B1 | Constant domains (double underlined) |
| (CDX-585) | scFv (broken underlined) |
| HC nucleic acids | underlined: Linkers (bolded and italicized) |
| | modified bases (bolded) |
| | CAAGTACAGCTCGTGCAATCTGGCGCCGAGGTAGTTAAACCTGGAGCATCCGTCAA |
| | AATTAGCTGTAAGGCAAGTGGCTACACTTTCACCGATTACGTCATGCACTGGGTGCG |
| | GCAGGCCCCCGGACAGAGTCTGGAGTGGATGGGCGTAATATCAACCTACCATATAC |
| | CCGCCGTGTACAATCAGAAATTTAAGGGTAAGGCAACCATGACCGTTGACACCTCCA |
| | CCTCTACAGTCTATCTGGAATTGTCTTCCCTAAGATCAGAGGACACTGCCGTCTACTA |
| | CTGTGCCCGCGAAGTTTATGGAGAGTCATATTACTTCGATGTTTGGGGTCAGGGAAC |
| | TACAGTAACTGTGAGCAGCGCTAGCACAAAGGGCCCTTCCGTGTTTCCACTGGCTCC |
| | CAGCTCTAAGTCCACCAGCGGAGGAACAGCCGCTCTGGGCTGTCTGGTGAAGGACT |
| | ATTTCCCAGAGCCCGTGACCGTGAGCTGGAACTCTGGCGCCCTGACCAGCGGAGTG |
| | CATACATTTCCTGCTGTGCTGCAGTCCAGCGGCCTGTACTCTCTGTCTTCCGTGGTGA |
| | CCGTGCCAAGCTCTTCCCTGGGCACCCAGACATATATCTGCAACGTGAATCACAAGC |
| | CATCCAATACAAAGGTGGACAAGAAGGTGGAGCCCAAGAGCTGTGATAAGACCCAT |
| | ACATGCCCCCCTTGTCCTGCTCCAGAGGCTCAGGGAGGACCATCCGTGTTCCTGTTTC |
| | CACCCAAGCCTAAGGACACCCTGTACATCACAAGGGAGCCAGAGGTGACCTGCGTG |
| | GTGGTGGACGTGAGCCACGAGGATCCCGAGGTGAAGTTCAACTGGTACGTGGATG |
| | GCGTGGAGGTGCATAATGCCAAGACAAAGCCAAGGGAGGAGCAGTACAATAGCAC |
| | CTATCGGGTGGTGTCTGTGCTGACAGTGCTGCACCAGGACTGGCTGAACGGCAAGG |
| | AGTACAAGTGCCAGGTGTCTAATAAGGCCCTGCCCGCTCCTATCGAGAAGACCATCT |
| | CCAAGGCCAAGGGCCAGCCTAGGGAGCCACAGGTGTACACACTGCCTCCAAGCCGG |
| | GACGAGCTGACCAAGAACCAGGTGTCTCTGACATGTCTGGTGAAGGGCTTCTATCCC |
| | TCTGATATCGCTGTGGAGTGGGAGTCCAATGGCCAGCCTGAGAACAATTACAAGAC |
| | CACACCCCCTGTGCTGGACTCCGATGGCAGCTTCTTTCTGTATTCCAAGCTGACCGTG |
| | GATAAGAGCAGGTGGCAGCAGGGCAACGTGTTTTCTTGTTCCGTGATGCATGAGGC |
| | 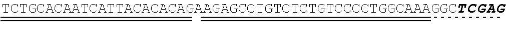 |
| | 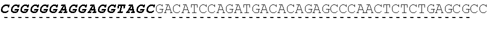 |
| | 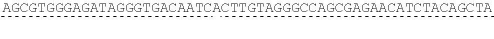 |
| | 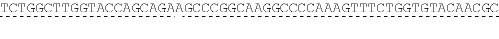 |
| | 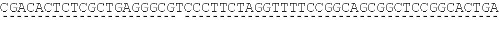 |
| | 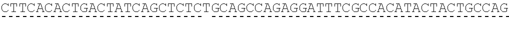 |
| | 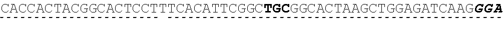 |
| | 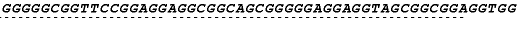 |
| | 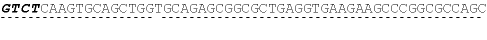 |
| | 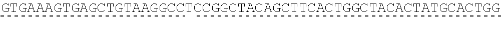 |
| | 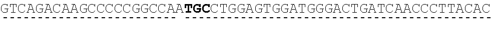 |
| | 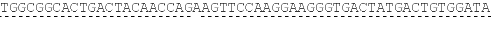 |
| | 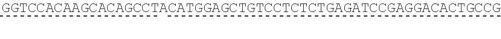 |
| | 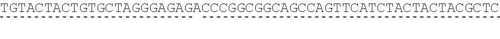 |
| | 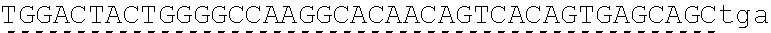 |

### EQUIVALENTS

Those skilled in the art will recognize or be able to ascertain using no more than routine experimentation, many equivalents of the specific embodiments of the invention described herein. Such equivalents are intended to be encompassed by the following claims.

We claim:

1. A bispecific construct comprising an anti-ILT4 binding domain linked to an anti-PD-1 binding domain, wherein:
(a) the anti-ILT4 binding domain comprises:
(i) a heavy chain variable region CDR1 amino acid sequence selected from the consensus sequence: G Y T (I,M) H (SEQ ID NO: 21), or conservative sequence modifications thereof;
(ii) a heavy chain variable region CDR2 amino acid sequence as set forth in SEQ ID NO:3, or conservative sequence modifications thereof;
(iii) a heavy chain variable region CDR3 amino acid sequence selected from the consensus sequence: E R P G G S Q F I Y Y Y (P,A) (M,L) D Y (SEQ ID NO:22), or conservative sequence modifications thereof;
(iv) a light chain variable region CDR1 amino acid sequence selected from the consensus sequence: R A S (A,E) NI Y S Y L A (SEQ ID NO: 23), or conservative sequence modifications thereof;
(v) a light chain variable region CDR2 amino acid sequence selected from the consensus sequence: N A (I,D) TL A E (SEQ ID NO: 24), or conservative sequence modifications thereof; and
(vi) a light chain variable region CDR3 amino acid sequence as set forth in SEQ ID NO:8, or conservative sequence modifications thereof; and
(b) the anti-PD-1 binding domain comprises:
(i) heavy chain variable region CDR1, CDR2 and CDR3 as set forth in SEQ ID NOs: 31, 36, and 41, respectively, or conservative sequence modifications thereof, and light chain variable region CDR1, CDR2 and CDR3 as set forth in SEQ ID NOs: 46, 51, and 56, respectively, or conservative sequence modifications thereof; or
(ii) heavy chain variable region CDR1, CDR2 and CDR3 as set forth in SEQ ID NOs: 69, 74, and 79, respectively, or conservative sequence modifications thereof, and light chain variable region CDR1, CDR2 and CDR3 as set forth in SEQ ID NOs:
84, 89, and 94, respectively, or conservative sequence modifications thereof.

2. The bispecific construct of claim 1, wherein:
(a) the anti-ILT4 binding domain comprises:
(i) heavy chain variable region CDR1, CDR2 and CDR3 as set forth in SEQ ID NOs: 11, 13, and 15, respectively, or conservative sequence modifications thereof, and light chain variable region CDR1, CDR2 and CDR3 as set forth in SEQ ID NOs: 16, 17, and 18, respectively, or conservative sequence modifications thereof, or
(ii) heavy chain variable region CDR1, CDR2 and CDR3 as set forth in SEQ ID NOs: 1, 3, and 5, respectively, or conservative sequence modifications thereof, and light chain variable region CDR1, CDR2 and CDR3 as set forth in SEQ ID NOs: 10, 11, and 12, respectively, or conservative sequence modifications thereof; and light chain variable region CDR1, CDR2 and CDR3 as set forth in SEQ ID NOs: 6, 7, and 8, respectively, or conservative sequence modifications thereof; and
(b) the anti-PD-1 binding domain comprises:
(i) heavy chain variable region CDR1, CDR2 and CDR3 as set forth in SEQ ID NOs: 31, 36, and 41, respectively, or conservative sequence modifications thereof, and light chain variable region CDR1, CDR2 and CDR3 as set forth in SEQ ID NOs: 46, 51, and 56, respectively, or conservative sequence modifications thereof; or
(ii) heavy chain variable region CDR1, CDR2 and CDR3 as set forth in SEQ ID NOs: 69, 74, and 79, respectively, or conservative sequence modifications thereof, and light chain variable region CDR1, CDR2 and CDR3 as set forth in SEQ ID NOs: 84, 89, and 94, respectively, or conservative sequence modifications thereof.

3. The bispecific construct of claim 1 or 2, wherein:
(a) the anti-ILT4 binding domain comprises heavy chain variable region CDR1, CDR2 and CDR3 as set forth in SEQ ID NOs: 11, 13, and 15, respectively, and light chain variable region CDR1, CDR2 and CDR3 as set forth in SEQ ID NOs: 16, 17, and 18, respectively; and
(b) the anti-PD-1 binding domain comprises heavy chain variable region CDR1, CDR2 and CDR3 as set forth in SEQ ID NOs: 31, 36, and 41, respectively, and light chain variable region CDR1, CDR2 and CDR3 as set forth in SEQ ID NOs: 46, 51, and 56, respectively.

4. The bispecific construct of any one of claims 1-3, wherein
(a) the anti-ILT4 binding domain comprises:
(i) a heavy chain variable region amino acid sequence as set forth in SEQ ID NO: 19, or a sequence at least 95% identical thereto, and a light chain variable region amino acid sequence as set forth in SEQ ID NO:20, or a sequence at least 95% identical thereto; or
(ii) a heavy chain variable region amino acid sequence as set forth in SEQ ID NO: 9, or a sequence at least 95% identical thereto, and a light chain variable region amino acid sequence as set forth in SEQ ID NO:10, or a sequence at least 95% identical thereto; and
(b) the anti-PD-1 binding domain comprises:
(i) a heavy chain variable region amino acid sequence as set forth in SEQ ID NO: 59, or a sequence at least 95% identical thereto, and a light chain variable region amino acid sequence as set forth in SEQ ID NO:60, or a sequence at least 95% identical thereto; or
(ii) a heavy chain variable region amino acid sequence as set forth in SEQ ID NO: 61, or a sequence at least 95% identical thereto, and a light chain variable region amino acid sequence as set forth in SEQ ID NO:62, or a sequence at least 95% identical thereto.

5. The bispecific construct of any one of claims 1-4, wherein the anti-ILT4 binding domain comprises a heavy chain variable region comprising SEQ ID NO:19 and a light chain variable region comprising SEQ ID NO:20

6. The bispecific construct of any one of claims 1-4, wherein the anti-ILT4 binding domain comprises a heavy chain variable region comprising SEQ ID NO:9 and a light chain variable region comprising SEQ ID NO:10.

7. The bispecific construct of any one of claims 1-6, wherein the anti-PD-1 binding domain comprises a heavy chain variable region comprising SEQ ID NO: 59 and a light chain variable region comprising SEQ ID NO:60.

8. A bispecific construct comprising an anti-ILT4 binding domain linked to an anti-PD-1 binding domain, wherein:
(a) the anti-ILT4 binding domain comprises a heavy chain variable region comprising SEQ ID NO: 19 and a light chain variable region comprising SEQ ID NO:20; and
(b) the anti-PD-1 binding domain comprises a heavy chain variable region comprising SEQ ID NO:59 and a light chain variable region comprising SEQ ID NO:60.

9. A bispecific construct comprising an anti-ILT4 binding domain linked to an anti-PD-1 binding domain, wherein:
(a) the anti-ILT4 binding domain comprises a heavy chain variable region comprising SEQ ID NO:9 and a light chain variable region comprising SEQ ID NO: 10; and
(b) the anti-PD-1 binding domain comprises a heavy chain variable region comprising SEQ ID NO:59 and a light chain variable region comprising SEQ ID NO:60.

10. A bispecific construct comprising an anti-ILT4 binding domain linked to an anti-PD-1 binding domain, wherein:
(a) the anti-ILT4 binding domain comprises a heavy chain variable region comprising SEQ ID NO:19 and a light chain variable region comprising SEQ ID NO:20; and
(b) the anti-PD-1 binding domain comprises a heavy chain variable region comprising SEQ ID NO:61 and a light chain variable region comprising SEQ ID NO:62.

11. A bispecific construct comprising an anti-ILT4 binding domain linked to an anti-PD-1 binding domain, wherein:
(a) the anti-ILT4 binding domain comprises a heavy chain variable region comprising SEQ ID NO:9 and a light chain variable region comprising SEQ ID NO:10; and
(b) the anti-PD-1 binding domain comprises a heavy chain variable region comprising SEQ ID NO:61 and a light chain variable region comprising SEQ ID NO:62.

12. The bispecific construct of any one of the preceding claims wherein the anti-PD-1 binding domain further comprises a human IgG1 constant domain.

13. The bispecific construct of any one of the preceding claims, wherein the anti-ILT4 binding domain is linked to the C-terminus of the heavy chain of the anti-PD-1 binding domain.

14. The bispecific construct of any one of the preceding claims, wherein the anti-ILT4 binding domain is a scFv.

15. The bispecific construct of any one of the preceding claims, wherein the anti-ILT4 binding domain and the anti-PD-1 binding domain are genetically fused.

16. The bispecific construct of any one of claims 1-14, wherein the anti-ILT4 binding domain and the anti-PD-1 binding domain are chemically conjugated.

17. A bispecific construct comprising an anti-PD-1 binding domain linked to an anti-ILT4 scFv, wherein:
(a) the anti-ILT4 scFv comprises heavy chain variable region CDR1, CDR2 and CDR3 as set forth in SEQ ID NOs: 11, 13, and 15, respectively, and light chain variable region CDR1, CDR2 and CDR3 as set forth in SEQ ID NOs: 16, 17, and 18, respectively; and
(b) the anti-PD-1 binding domain comprises heavy chain variable region CDR1, CDR2 and CDR3 as set forth in SEQ ID NOs: 31, 36, and 41, respectively, and light chain variable region CDR1, CDR2 and CDR3 as set forth in SEQ ID NOs: 46, 51, and 56, respectively, and a human IgG1 constant domain.

18. The bispecific construct of claim 17, wherein:
(a) the anti-ILT4 scFv comprises a heavy chain variable region comprising SEQ ID NO: 19 and a light chain variable region comprising SEQ ID NO:20; and
(b) the anti-PD-1 binding domain comprises a heavy chain variable region comprising SEQ ID NO:59 and a light chain variable region comprising SEQ ID NO:60.

19. A bispecific construct comprising an anti-PD-1 binding domain linked to an anti-ILT4 scFv, wherein:
(a) the anti-ILT4 scFv comprises heavy chain variable region CDR1, CDR2 and CDR3 as set forth in SEQ ID NOs: 11, 13, and 15, respectively, and light chain variable region CDR1, CDR2 and CDR3 as set forth in SEQ ID NOs: 16, 17, and 18, respectively; and
(b) the anti-PD-1 binding domain comprises heavy chain variable region CDR1, CDR2 and CDR3 as set forth in SEQ ID NOs: 69, 74, and 79, respectively, and light chain variable region CDR1, CDR2 and CDR3 as set forth in SEQ ID NOs: 84, 89, and 94, respectively, and a human IgG1 constant domain.

20. The bispecific construct of claim 17, wherein:
(a) the anti-ILT4 scFv comprises a heavy chain variable region comprising SEQ ID NO: 19 and a light chain variable region comprising SEQ ID NO:20; and
(b) the anti-PD-1 binding domain comprises a heavy chain variable region comprising SEQ ID NO:61 and a light chain variable region comprising SEQ ID NO:62.

21. A bispecific construct comprising an anti-PD-1 binding domain linked to an anti-ILT4 scFv, wherein:
(a) the anti-ILT4 scFv comprises heavy chain variable region CDR1, CDR2 and CDR3 as set forth in SEQ ID NOs: 1, 3, and 5, respectively, and light chain variable region CDR1, CDR2 and CDR3 as set forth in SEQ ID NOs: 6, 7, and 8, respectively; and
(b) the anti-PD-1 binding domain comprises heavy chain variable region CDR1, CDR2 and CDR3 as set forth in SEQ ID NOs: 31, 36, and 41, respectively, and light chain variable region CDR1, CDR2 and CDR3 as set forth in SEQ ID NOs: 46, 51, and 56, respectively, and a human IgG1 constant domain.

22. The bispecific construct of claim 21, wherein:
(a) the anti-ILT4 scFv comprises a heavy chain variable region comprising SEQ ID NO: 9 and a light chain variable region comprising SEQ ID NO:10; and
(b) the anti-PD-1 binding domain comprises a heavy chain variable region comprising SEQ ID NO:59 and a light chain variable region comprising SEQ ID NO:60.

23. A bispecific construct comprising an anti-PD-1 binding domain linked to an anti-ILT4 scFv, wherein:
(a) the anti-ILT4 scFv comprises heavy chain variable region CDR1, CDR2 and CDR3 as set forth in SEQ ID NOs: 1, 3, and 5, respectively, and light chain variable region CDR1, CDR2 and CDR3 as set forth in SEQ ID NOs: 6, 7, and 8, respectively; and
(b) the anti-PD-1 binding domain comprises heavy chain variable region CDR1, CDR2 and CDR3 as set forth in SEQ ID NOs: 69, 74, and 79, respectively, and light chain variable region CDR1, CDR2 and CDR3 as set forth in SEQ ID NOs: 84, 89, and 94, respectively, and a human IgG1 constant domain.

24. The bispecific construct of claim 23, wherein:
(a) the anti-ILT4 scFv comprises a heavy chain variable region comprising SEQ ID NO: 9 and a light chain variable region comprising SEQ ID NO: 10; and
(b) the anti-PD-1 binding domain comprises a heavy chain variable region comprising SEQ ID NO:61 and a light chain variable region comprising SEQ ID NO:62.

25. The bispecific construct of claim 19, wherein the anti-PD-1 binding domain and the anti-ILT4 scFv comprise heavy and light chain sequences as set forth in SEQ ID NOs: 64 and 63, respectively.

26. The bispecific construct of claim 19, wherein the anti-PD-1 binding domain and the anti-ILT4 scFv heavy and light chains are encoded by the nucleotide sequences as set forth in SEQ ID NOs: 66 and 65, respectively.

27. A composition comprising the bispecific construct of any one of claims 1 to 26 and a pharmaceutically acceptable carrier.

28. The composition of claim 27, further comprising one or more therapeutic agents.

29. The composition of claim 27 or 28, wherein the therapeutic agent is another antibody.

30. The composition of any one of claims 27 to 29, wherein the antibody is an anti-PD-L1 and/or an anti-CTLA-4 antibody.

31. A kit comprising the bispecific construct of any one of claims 1 to 26, or the composition of any one of claims 27 to 30, and instructions for use.

32. A method of activating macrophages comprising contacting macrophages with the bispecific construct of any one of claims 1 to 26, or the composition of any one of claims 27 to 30.

33. A method for inducing or enhancing an immune response in a subject comprising administering to the subject the bispecific construct of any one of claims 1 to 26, or the composition of any one of claims 27 to 30, in an amount effective to induce or enhance an immune response in the subject.

34. A method for treating a condition or disease in a subject, the method comprising administering to the subject the bispecific construct of any one of claims 1 to 26, or the composition of any one of claims 27 to 30, in an amount effective to treat the condition or disease.

35. The method of claim 34, wherein the subject suffers from a condition or disease in which stimulation of an immune response is desired.

36. The method of claim 34 or 35, wherein the condition or disease is cancer.

37. The method of claim 36, wherein the cancer is skin cancer, colorectal cancer, ovarian cancer, renal cell carcinoma, head and neck squamous cell carcinoma, breast cancer, lung cancer, bladder cancer, prostate cancer, melanoma, gynecological cancers, sarcoma, lymphoma, or glioblastoma.

38. A method of treating a tumor in a subject, the method comprising administering to the subject the bispecific construct of any one of claims 1 to 26, or the composition of any one of claims 27 to 30, in an amount effective to treat the tumor.

39. The method of claim 38, wherein the tumor expresses ILT4, HLA-G, HLA class I, angiopoietin like 2, Nogo, an ILT4 ligand, PD-L1, or PD-L2.

40. The method of any one of claims 33 to 39, further comprising administering one or more therapeutic agents to the subject.

41. The method of claim 40, wherein the therapeutic agent is another antibody.

42. The method of claim 41, wherein the antibody is an anti-PD-L1 and/or an anti-CTLA-4 antibody.

43. The method of any one of claims 40 to 42, wherein the bispecific construct and the one or more therapeutic agents are administered concurrently.

44. The method of any one of claims 40 to 42, wherein the bispecific construct and the one or more therapeutic agents are administered sequentially.

45. Use of the bispecific construct of any one of claims 1 to 26, or the composition of any one of claims 27 to 30, in the manufacture of a medicament for treatment of cancer.
